(19) United States
(12) Patent Application Publication
(10) Pub. No.: US 2001/0019821 A1
(43) Pub. Date: Sep. 6, 2001

(54) Method for manufacturing and detecting and normalizing HIV for rapid analysis

(76) Inventor: Jack V. Smith, Arden, NC (US)

Correspondence Address: Jack V. Smith, P.O. Box 156, Arden, NC, 28704, US

(21) Appl. No.: 09/843,422

(22) Filed: Apr. 25, 2001

Related U.S. Application Data
(62) Division of application No. 09/283,318, filed on Mar. 31, 1999.

(51) Int. Cl. C12Q 1/70; G01N 33/543

(52) U.S. Cl. 435/5; 436/518; 422/50; 422/55; 436/523

(57) ABSTRACT

Method for analyzing a sample using an aqueous liquid reagent to determine the concentration of HIV antibody in an individual's random urine sample in order to determine if the individual's exposure to the HIV virus, and normalizing or correcting this assay value with the sample's creatinine, cystatin C, or specific gravity concentration.

This is a division of Ser. No. 09/283,318, Filed Mar. 31. 1999

### BACKGROUND OF THE INVENTION

1. Field of the Invention

The present invention is a method for the use of purified antigen from a biological or other source that is specifically targeted by HIV (human immunodeficiency virus) antibodies or with the use of an antibody to an HIV antibody. These antigens and antibodies are then used to develop specific particles and markers used in the detection of HIV antibodies. These afore mentioned proteins (antigens and antibodies to HIV) are referred to now as “HIV markers”. The HIV markers are then used to assay for the presence of HIV antibodies via rapid, dipstick or lateral flow methodology (liquid/solid phase assay) and automated, liquid methodology (liquid/liquid phase assay) on 24 hour urine or random blood collection samples. Alternatively, these assays can utilize random, or “spot” urine samples, if creatinine, cystatin C or specific gravity is also determined on the sample and then used to “normalize” the HIV antibody test value.

2. Description of the Related Art

Acquired Immune Deficiency Syndrome, or AIDS, was first reported in the early 1980's. The human immunodeficiency virus (HIV) that causes AIDS was also discovered in the early 1980s. By 1997 HIV had affected over 30.6 million people worldwide and HIV newly affects over 6 million people every year. AIDS is a fatal disease caused by HIV and attacks the individual's immune system, gradually leaving the individual helpless against opportunistic infections and diseases that cause death. The most common is Pneumocystis carinii pneumonia (PCP), a parasitic infection of the lungs, and a type of cancer known as Kaposi's sarcoma (KS).

With no cure in site abstinence could prevent thousands possibly millions from becoming exposed to the virus. The fate of millions of people will largely depend on the ability of people to change their behavior patterns when it comes sex without protection than what science can do. The present art provides a method for millions of individuals to test themselves in the privacy of their homes. Millions of people have avoided the test because of the social implication that a positive AIDS test result presents. A positive test is reported to the public health agency, the workers in a physician's office know if the patients have AIDS. There is definitely a stigma attached to anyone that has AIDS. With the ability to self test without information leakage could lead to saving millions of people from exposure to AIDS. It is hoped that once the individual knows that they have AIDS that they would abstain from sex without protection for the partner.

A thorough search of patents and research revealed no relative art (i.e., prior art) showing any correlation to this technology. The search has included a search of the USPTO (United States Patent Office) database with no patents issued for the urine and blood testing for AIDS. The art of manual testing blood by the use of ELISA microscopic analysis aside, no chemical HIV detection test means even slightly similar to the present art has been described prior. However, the following art will be mentioned to further illustrate the novelty of the present art and the obvious advancement to the current art. The following patents, with the exception of do not mention the use of urine as the test matrix for detecting specific analytes of interest. It is known in the art that the urine matrix is very complex and consists of many urinary constituents which create strong buffering and interference problems (e.g. cannibal-like enzymes such as protease) that have to be overcome to provide a method that can be used for the general population with precision and accuracy. Simply because a technique can accommodate a liquid sample does not imply that it can be successfully used with any liquid test matrix. Such successful adaptation of test techniques to accurately deal with specific sample matrices aren't often “obvious” to any scientist.

U.S. Pat. No. 4,575,486, claims the detection of red and white blood cells coated with Tamm-Horsfall Protein (THP) for the purposes of trying to determine the origination of red and white blood cells. This patent claims to be able to do this by the use of an antibody to form antibody-antigen complexes that are detected by visual, fluorescent or radioactive techniques in a liquid reagent. This patent is fatally flawed and is of little if any clinical value because it is well known in the art that normal urine is supposed to be free of RBC's, WBC's and other cellular constituents and only a minute amount of THP coated RBC'S and WBC's ever enter the urinary tract. On the other hand, the vast majority of the RBC's and WBC's found in urine are due to infections of the bladder or urethra; these cells are not THP-coated thereby making the detection of THP-coated red and white blood cells improbable if not impossible by this method. This patent does not disclose a method for the determination of HIV antibodies in urine or any other matrix. This patent also fails to teach the use of ultra-violet or visible (i.e. UV-Vis) colorimetric, spectrophotometric, and reflectance techniques for the determination of HIV antibodies in urine utilizing dry chemistry dipsticks or lateral flow devices (LFD) for manual determination or aqueous reagent formulas compatible with an automated chemistry analyzer. This disclosure also fails to teach any means for determination of HIV using enzymatic, antigen/antibody, or chromagenic reaction.

Another patent, U.S. Pat. No. 3,961,039, is a stain for urinary sediments utilized on a glass slide. It does not teach a method of determining the presence of urinary antibodies that are indicative of HIV. It does not teach dipstick or LFD assay techniques. It also does not describe a liquid reagent that is compatible with automated chemistry analyzers. In fact, attempting to adapt its formulations to an automated analyzer would cause significant damage to the optics and test cuvettes. This patent fails to disclose the use of fluorescent, radioactive, UV-Vis, calorimetric, spectrophotometric, and reflectance techniques for the determination of HIV antibodies that are indicative of AIDS. This disclosure also fails to mention any modes of determination of AIDS by any measurable means including enzymatic, antibody-antigen, calorimetric, or other means.

Another patent, U.S. Pat. No. 4,446,232, is an enzyme immunoassay technique using detection zones for the determination of the presence of antibodies and does not teach or suggest the method of determining the presence of antibodies that are indicative of HIV antibodies in urine. It also fails to teach assay techniques using a dry chemistry dipstick or LFD, or a liquid reagent compatible with automated chemistry analyzers. This patent also fails to mention any use of ultra-violet, calorimetric, spectrophotometric and reflectance techniques for the determination of HIV antibodies in urine or any other matrix (i.e., blood, etc.). This disclosure also fails to mention any modes of determination of urinary antibodies that are indicative of HIV by any measurable detectable means such as enzymatic, antibody-antigen, calorimetric, or other chemical means.

Another patent, U.S. Pat. No. 4,786,589, is an immunoassay using formazan-prelabled reactants and does not teach or suggest the method of determining the presence of urine HIV antibodies by the use of a dry chemistry dipstick (DCD) or LFD, or a liquid reagent compatible with automated analyzers. This art requires the sample to be premixed with a labeled primary protein having a specific affinity for the analyte of interest. This patent does describe dipsticks. The pretreatment step, however, creates significant problems. This step makes application to an automated chemistry analyzer fruitless, because of the additional labor it requires. This step also makes it unacceptable for point-of-care and home use with DCD's and LFD's, because of the potential for errors. Again, this method fails to teach a method for the determination of AIDS. This patent also fails to teach the use of UV-Vis colorimetric, spectrophotometric, and reflectance techniques for the determination of HIV antibodies in urine or any other matrix. It also doesn't describe the use of DCD's or LFD's for manual determination, or aqueous reagents for use on automated chemistry analyzers. This disclosure also fails to teach a method for determination of urinary HIV antibodies via enzymatic, antibody-antigen, colorimetric, or other chemical means.

Another patent, U.S. Pat. No. 3,603,957, teaches the use of assay test strips but again fails to disclose a method for the determination of HIV antibodies in urine. This patent also fails to teach the use of UV-Vis, colorimetric, spectrophotometric and reflectance techniques for the determination of HIV antibodies in urine; it also doesn't teach the use of DCD's or LFD's or aqueous formula for use on an automated instrument. This disclosure also fails to teach a method for determination of urinary HIV antibodies via enzymatic, antibody-antigen, calorimetric, or other means.

In the literature and prior art, techniques such as ELISA and other methods have been used to detect certain HIV markers in blood, however, these methods have no relevant bearing on the present device. ELISA is a technique that coats a micro-titer well plate with antibody for the particular analyte of interest. This immobilized method has no similarity or relevance to the DCD, LFD, or aqueous reagent for automated instruments. It would be impossible to grind up a micro-titer well plate and somehow liquefy it for use on an automated instrument for the quantitative determination of urinary osteoporosis antigens. It is also improbable to apply this logic to a dipstick or LFD assay technique. The afore mentioned techniques, along with two-site immunochemiluminometric techniques, have no bearing on the present device for obvious reasons. For instance, the ELISA is an immobilized method, and the reaction mixture cannot, therefore, be moved from one area to another (like a carrier-free aqueous reagent which is transferred from a reagent container to a reaction cuvette of an automated chemistry analyzer).

The two-site immunochemiluminometric techniques can assay for target proteins by pretreating each sample with a specific binding protein; the bound and free fractions must then be separated by another antibody-antigen reaction, and then linked to magnetic particles and measured by some means. This process offers numerous obstacles. First, this technique is very time consuming, and not applicable to current chemistry automation or manual one-step DCD's and LFD's. Obviously, the present device represents a significant advancement over these older techniques including ELISA, microscopic analysis, electrophoresis, two-site immunochemiluminometrics, immunofluorescent staining, zone detection, slide staining, and multiple detection layers.

Again, compare ELISA versus an aqueous reagent applied to an automated chemistry analyzer for quantitative analysis. The former takes up to an two hours or more. The latter yields results in a few minutes along with quality controls to validate the accuracy of the data.

It is well known in the art, that these two methodologies are very time consuming and labor intensive, and require hours to complete analysis on a single sample; they also require complex, and expensive instrumentation. As a whole ELISA and HPLC are not effective assays for high volume testing, small clinics or doctors' offices, or home testing because of costs, sophisticated equipment and associated skill required, and analysis time. In particular, HPLC, is very complex and requires many assay steps including sample clean up, derivatization, purification, and which result in low variable yields. ELISA requires even more steps prior analysis, including preparation of a micro titer plate, predilution, and numerous serial dilution's, PBS (phosphate buffered saline) pre-incubation, incubation with a secondary antibody, addition of a color reagent, interruption of the color reaction, and finally the absorbance is determined. Obviously, these multi-step assays are very tedious and time consuming, and require significant analytical skills.

As the foregoing illustrates there is a need in the art for rapid analysis of HIV antibodies in urine and other matrices to accurately determine if a test subject has been exposed to the HIV virus and has the potential to acquire AIDS. These assays should simple and inexpensive to perform in order to make them widely available.

Assay techniques which fill this description include dry chemistry dipsticks (DCD), lateral flow devices (LFD), and aqueous liquid reagents compatible with automated chemistry analyzers (ACA). The HIV virus as known in the art is directly related to AIDS. The presence of the HIV virus in a test subject indicates a high level of confidence that the subject will acquire AIDS. The presence of the HIV antibody in urine therefore indicates that the test subject has been exposed to the HIV virus and has the potential to exhibit and have the AIDS disease. The present device provides an inexpensive, readily available, rapid analysis for HIV, therefore, can prevent the injury and loss of life due to this disease. Early detection and initiation of treatment is critical, and this device makes that task simple and inexpensive, because a DCD or LFD assay can be performed at home or in the doctor's office. Another important aspect to the present device is its utility in evaluation of the treatment regimen for the disease. The present art permits the physician to determine if the patient is responding to therapy routinely and painlessly without having to tamper with infectious blood. Current art requires very expensive analysis and large amount of test time to effectively determining the presence of HIV.

This faster window of evaluation allows the physician to alter treatment as needed. It should also be noted that AIDS treatment is very expensive, and very painful for the patient. The rising costs of health care require that we do everything possible to improve the efficacy of all healthcare intervention. The present art provides a method for the general consumer (patient) to save money and still receive the health care needed by providing a test result for dollars at home or in the clinic versus the current art which costs hundreds of dollars. Ultimately this could save the consumer, nation and world economy millions of dollars. The clinical treatment of AIDS is very expensive and time consuming as is well known in the art. Early detection of this disease, and optimization of treatment is imperative to save dollars and lives.

### SUMMARY OF THE INVENTION

Rapid, one-step home and physician's office testing currently takes the form of DCD's and LFD's. These devices consist of absorbent carriers, usually paper, which has been impregnated with all of the chemicals needed for the detection reaction. After dipping the DCD into a body fluid, or adding a drop of fluid to the test pad, a color reaction takes place. Because of the importance of achieving rapid results dipsticks have been developed to detect various disease markers in body fluids. Another rapid test device, the LFD, is very similar to a dipstick in principle. This device combines the DCD with some aspects of thin layer chromatography (TLC) principles. After dipping one end of the LFD into a sample, the urine migrates up the paper (or absorbent material) to the reactive sites containing reagents (reactive ingredients). The urine constituents react with the assay reagents during the migration process and yield visible results. Automated liquid chemistry analysis utilizes aqueous reagent mixtures used in conjunction with automated chemistry analyzers. This assay system utilizes microliter amounts of reagents and samples and produces accurate results on hundreds or thousands of samples per hour with minimal labor (e.g. 1 technologist per instrument).

For the detection of HIV antibodies in urine, the sensitivity of the test is of decisive importance and, furthermore it is also desirable. The dipstick test or LFD has a qualitative to quantitative sensitivity range of approximately 10.0 fmol/L or less of HIV antibodies to 1,000,000 fmol/L of HIV antibodies or greater. On the other hand, the automated liquid test has a sensitivity range of 1.0 fmol/L to 1,000,000 fmol/L or greater. The HIV antibodies that are targeted in this are specific for the HIV antigen (virus) that causes AIDS. The present art can have sensitivity and detection limits in the zeptomole range (which is one zeptomole=1000 attomoles or 1,000,000 femtomoles).

Examination of patents and published research reveal no relative art (i.e., prior art) even slightly resembling this technology. Other than that discussed above, currently utilized methodology is clearly inferior to this new art. No chemical test means has been described prior to this disclosure which can perform the tasks this new art can.

Briefly stated, the present invention relates to test devices for measurement of HIV antibodies in urine but could also work in other matrices such as blood, saliva, or other fluids that come from the human body of other animals, and the procedures for making said test means. This invention is in the field of clinical diagnostics. More specifically, this invention provides dry chemistry dipsticks (DCD's or on-site test modules), thin layer chromatographic dry chemistry technology (LFD's), and aqueous, liquid chemistry reagents that quantitate HIV antibodies to determine is the test subject has been exposed to the HIV virus that causes AIDS on biological samples (e.g. urine, serum, and blood). This new art can utilize aqueous, biological specimens including urine, saliva, sweat extracts, blood, and serum. In addition, this invention provides a unique method for HIV antibody measurement utilizing rapid test devices including the DCD, and LFD thereby enabling in-home testing through over-the-counter (OTC) sales. This is an enormous advancement in the art. These advances and improvements of the present device over the prior art provides the health care testing industry with powerful new clinical and diagnostic tools.

This invention eliminates the need for the costly HPLC, and/or ELISA plate testing and the concomitant long term testing requirement to adequately evaluate treatment efficacy (i.e. 6 to 12 months) currently necessary. This invention also improves the sensitivity, specificity, accuracy, and economics of analysis by applying it principles to DCD's, LFD's, and aqueous, liquid chemistry reagents. Note, the previous art taken as a whole, does not enable an effective HIV assay method capable of utilizing the dry chemistry dipstick format for several reasons; these include sensitivity, specificity, accuracy and lack of stability of the procedures, incompatibility of the prior methods with these assay requirements, safety hazards, and susceptibility to interference.

This new art described herein fills two key needs in two diverse arenas. The first area of need involves physicians' offices and their ability to diagnose AIDS through the use of dry chemistry HIV assay on urine and not blood as in the prior art. This dipstick/LFD assay is ideal for this application, because it requires no sophisticated equipment, or training, is much less expensive, and provides immediate results. These test devices can also be utilized by laypersons at home, or in countries in which sophisticated lab work is not possible. The second arena of advantage lies in large, high volume, reference labs. These facilities typically serve as regional test centers, and perform large numbers of tests each day. Such labs would use automated chemistry analyzers in conjunction with aqueous, liquid reagents to reduce technician time, lower cost of testing, and test large numbers of samples in very short time periods. The most important aspect is the present art's ability to assay for HIV without the use of blood or blood products which are highly infectious.

The reactants that target and react with the HIV antibody can include anti-anti-HIV (I or II), anti-HIV (I or II), HIV antigens (I or II), and HIV aptamers. All of these reactants will produce a detectable response in the presence of HIV antibody.

The present invention relates to a method that can be used by two different techniques. One technique employs dry chemistry technology for DCD'S and LFD'S as outlined above. A second technique employs an aqueous reagent compatible with automated chemistry analyzers currently available to medical labs. As indicated above both of these techniques can be used to measure for HIV antibodies allowing the determination of AIDS. The advantages of the dry chemistry technique include ease of use, semi-quantitative or quantitative results, low cost, and technical improvements (e.g. increased sensitivity, specificity, and accuracy, and reduced interference); no one has SUCCESSFULLY ADAPTED any of the prior art for HIV testing to dry chemistry applications. This technology is manufactured by impregnating onto absorbent paper the chemical constituents which have been dissolved in a liquid format, evaporating the liquid, and mounting this “test paper” on a sturdy plastic handle.

The advantages of the liquid reagent technique are true quantitation, reduced cost per test, technical improvements (e.g. increased sensitivity, specificity, and accuracy, and reduced interference's), expanded range of detection (lower and upper limits), and compatibility to chemistry analyzers thereby permitting assay of hundreds of specimens per hour with minimal labor (e.g. one technologist can operate one or two analyzers which are doing twelve or more different assays on up to one thousand samples/hour).

This new art is composed of an indicator(s) (i.e. colorimetric, enzymatic, fluorescent, turbidimetric, radioimmunologic, antigen-antibody, ion-exchange, or ionic), and buffers. Interference-removing compounds may also be included but are not required for the assay to work effectively. The present art's assay for HIV may be further enhanced by the use of a creatinine, cystatin C, or specific gravity assay performed on the same urine sample. This enhancement permits the use of a random or spot urine instead of collection of a 24 hour sample. The creatinine, cystatin C, or specific gravity value is used to “normalize” or correct the test result for diurnal variations. For example, if the urine were dilute the HIV value would be low and should be adjusted upward to a higher value. And if the urine were concentrated the HIV value would be high and should be lowered. The objective of this procedure is to determine how much of the marker protein is excreted per day. It is known in the art that creatinine and cystatin C as well as other markers are steady state components of human urine and can be used as a reliable source to determine urine concentration. Specific gravity, and osmolality can also provide the same information. Research has revealed no relative prior art to this invention thereby eliminating the obviousness of this novel invention. The current art bears no relation to that which is described herein.

The first method for measurement of HIV utilizes monoclonal anti-anti-HIV conjugated to glucose-6-phosphate dehydrogenase an indicator/substrate sensitive to dehydrogenase activity. The assay is dependent upon the concentration of the HIV antibody and its corresponding effect on the bound dehydrogenase. The use of anti-anti-HIV and glucose-6-phosphate are merely illustrative for this unique invention and other possibilities are possible as will be explained.

A second method includes color producing indicator compounds that yield ultra-violet or visible color and can be bound to an HIV antigen or HIV aptamer (aptamers are nucleic acid molecules that bind specific ligands, like antibodies aptamers have high affinities and specificity's for targets such as HIV antigens and antibodies) that are specific for HIV antibodies. These color-producing compounds can be bound to the antigen or aptamer via covalent or ionic bonding to form antigen-indicator complex. Examples of indicators that yield a detectable colorimetric response, and may be used for this purpose include horseradish peroxidase (HRP), tetramethylbenzidine (TMB) para-nitroaniline, glucose-6-phoshpate dehydrogenase (G6PDH), alkaline phosphatase (AP), fluorescein (FITC), tetramethyl rhodamine isothiocynate (TRITC), Biotin, phycoerytherin (PHYCO), and naphthylamine (see examples for additional compounds). When this antigen-indicator complex contacts the antigen's antibody the complex's bond is fractured thereby yielding the colored indicator or the chromogen portion of the complex is activated and will react with other substrate in solution with the complex. In some cases additional reactants may be required to combine with the released compound to produce a colored product. For example, the HIV antigen/p-nitroanilide compound would yield the HIV antibody-HIV antigen complex and p-nitroaniline; this latter compound will yield a yellow color. In the case of naphthylamine the additional reactants would include sodium nitrite and N-1-naphthylethylenediamine. This secondary reaction would produce a blue color. The antigen-indicator complexes are obtained via standard organic synthesis. These assays can be competitive binding assays. The antigen-indicator is mixed with sample. This solution will attack any endogenous HIV antibody in the sample. The rate at which color is generated over a fixed amount of time is determined, and compared to that produced by standards with known amounts of the antigen, to quantify the concentration of analyte in the unknowns. This quantification can be observed visually, or measured via spectrophotometer.

The third method requires an antibody specific for HIV antibody, such as anti-HIV-1 or anti-HIV-2. This immunoassay technique is based on competitive binding between free anti-HIV (HIV antibody) in the sample and anti-HIV antibody conjugated to an enzyme such as glucose-6-phosphate dehydrogenase (anti-HIV-G6PDH) for available anti-anti-HIV contained in part one of the reagent (R-1). The anti-anti-HIV in the R-1 is first mixed with sample enabling any anti-HIV present to bind to it. This R-1 solution also contains the substrate, glucose-6-phosphate (G6P), and a co-enzyme, Nicotinamide Adenine Dinucleotide (NAD). After incubation of the sample with the R-1, part two of the reagent, R-2, is added. This contains the enzyme and anti-HIV-glucose-6-phosphate dehydrogenase (anti-HIV-G6PDH) conjugate. If no analyte (anti-HIV) is present in the sample, the free anti-anti-HIV antibody in the reagent R-1 will bind to the anti-HIV conjugated to the G6PDH (e.g. Ntp-G6PDH) thereby inactivating the enzyme. As a result, the enzyme, G6PDH, is not able to react with the substrate, G6P, and the co-enzyme, NAD, and therefore no color change results as measured at 340 nm. If anti-HIV is present, the anti-anti-HIV antibody in R-1 will bind to it leaving the anti-HIV-G6PDH free to react with its substrate, G6P, and co-enzyme NAD; the NAD is converted to NADH during the reaction yielding a decrease in absorbance as measured spectrophotometrically at 340 nm. Obviously, enzyme reaction kinetics (or enzyme binding to the substrate) decrease proportionately to the amount of anti-HIV present in the sample, therefore, its concentration in the sample can be measured in terms of enzyme kinetics; the amount of color generated is inversely proportional to the amount of anti-HIV in the sample. This reagent system of the instant invention (liquid reagent) is intended for use on any automatic chemistry analyzers with open channel capability including Olympus AU 5000 series, Hitachi 700 series, and many others as well as DCD's or LFD's.

An example of the analysis procedure utilizing the reagent system of the instant invention described herein is as follows: the two components of the reagent composition (R-1 and R-2) are placed in the reagent compartment of the analyzer; samples, calibrators, and controls are aliquoted into sample cups which are then placed on the analyzer. An aliquot of 10 uL of each specimen is then pipetted into a single, discrete cuvette followed by the addition of 125 uL of the first reagent, R-1, and mixed; After a specified incubation time of five minutes, 125 uL of the second reagent, R-2, is added to the cuvettes, and mixed. A first spectrophotometer reading is then taken followed by a second after a specified incubation period (i.e. one minute for this example) at the specified wavelength (between 340 and 800 nm). The spectrophotometer readings are then recorded. In this instance the assay is read at 340 nm. The absorbance of samples, and controls are stored and then compared to a standard curve derived from the calibrators' absorbance; this comparison yields quantitative values for the unknowns and controls, which are printed on a report. This method will function for liquid, automated analysis, only. An indicator that yields a visible (measurable) color change is required for dry chemistry dipstick analysis. For example, inclusion of a tetrazolium indicator (e.g. nitro-blue tetrazolium) and an electron carrier (e.g. 1-methoxy-5-methylphenazium) will yield a color change in the visible spectrum. This color reaction could be utilized for DCD's and LFD's as well as in the aqueous, liquid reagent system. Another alternative for production of a visual color change would require substitution of G6PDH (conjugated to anti-HIV), its substrate, G6P, and NAD with Galactosidase and 5-bromo-6-chloro-3-indoxyl-beta-D-galactopyranoside will produce a magenta color that increases with increasing concentration of the target marker (e.g. anti-HIV). Another variation of the above methodology would utilize a fluorescent marker in place of the NAD, and could be measured using fluorescent spectroscopy.

Yet another variation of the immunoassay technique for analysis of HIV antibody in biological fluids utilizes particle-enhanced aggregation (PEA). An example of this technique includes an R-1 which contains antibody to the analyte of interest, anti-anti-HIV for example. The R-2 contains microparticles conjugated to HIV antigen (or to an HIV aptamer). The reagent's R-1 is mixed with sample. If anti-HIV is in the sample, it binds to the anti-anti-HIV antibody. The R-2 is then added. Any unbound antibody is then free to react with the HIV antigen conjugated to the microparticles. This reaction promotes formation of particle aggregates. As the aggregation reaction proceeds in the absence of free anti-HIV in the sample, the absorbance monitored increases spectrophotometrically. Conversely, the presence of anti-HIV diminishes the absorbance in proportion to the concentration of it in the sample. This assay can be monitored spectrophotometrically from 340 to 800 nm. Alternatively, antibody or antigen to anti-HIV can be chemically bound to the polystyrene microparticles. These antibody-microparticles or antigen-microparticles or aptamer-microparticles bind to any anti-HIV present in the sample, and in this process form aggregates. Therefore, the absorbance of the reaction mixture increases in proportion with the concentration of anti-HIV present. This absorbance change can be read between 340 and 800 nm. This same unique technology can be used for DCD's and LFD's.

The dry chemistry, on-site assay devices (DCD's) utilizing particle enhanced aggregation for analysis of HIV in biological fluids contain microparticles of uniform size, chemically coupled with antibody to one or more of the markers noted above (e.g. anti-anti-HIV). In the case of a static dipstick device, the microparticles are also conjugated with an indicator. If anti-HIV is present, the antibody-microparticles bind to it, and simultaneously displace the indicator; this results in the formation of color on the test pad. If no anti-HIV is present, no color forms. Obviously, the amount of color formed is proportional to the amount of anti-HIV present. When this assay model is adapted to liquid format, the color indicator is not required (but could be used). The microparticles which react with anti-HIV to form anti-HIV-antibody-microparticle complexes will spontaneously combine to form aggregates. The formation of said aggregates will cause an increase in absorbance. Therefore, absorbance (read between 340 and 800 nm) is directly proportional to the anti-HIV concentration which can then be correlated to bone loss.

Another type of on-site test methodology utilizing PEA technology combines thin layer chromatography with dry chemistry dipstick technology (i.e. LFD's). In this case, the microparticles are chemically coupled to an antibody against a specific analyte (e.g. anti-HIV) and are colored, but are not conjugated to an indicator. Sample mixes with the microparticles at the base or starting line of the LFD. If anti-HIV is present, it binds to the antibody-microparticle (anti-anti-HIV microparticle). This antibody-microparticle-HIV complex (i.e. anti-HIV-anti-anti-HIV-MP) then continues wicking up the strip past a result line (1st window) to the validation line (2nd window) which is composed of antibody to the antibody conjugated to the microparticle (e.g. anti-anti-anti-HIV) which is bound to the test strip via a protein. The antibody-microparticle-HIV complex then reacts (binds) to the anti-anti-anti-HIV antibody; the end result being a visible colored line formed by the colored microparticles in the second, or validation window. If anti-HIV is not present in the sample, the antibody-microparticles wick up the test strip until they reach the result (first) window in which anti-HIV has been bound to the paper. The antibody-microparticles (i.e. anti-anti-HIV-MP) then binds to the immobilized anti-HIV formning a colored line as a result of the colored particles. Please note, however, that antibody-microparticles need to exceed the quantity of anti-HIV bound to the strip in the result window. This excess of antibody-microparticles, therefore, continue migrating up the test strip to the validation window where they bind to the anti-anti-anti-HIV forming a visible colored line and confirms the test is complete. Note, therefore, that a colored line wIll form in the validation window in the case of a positive or negative result. On the other hand, no line will form in the result window in the case of a positive result. This technique can be further simplified by eliminating the antibody to anti-anti-HIV antibody in the validation window. Excess colored microparticles will still congregate at the top of the device thereby forming a visible line, and indicating completion of the test.

A final method for analysis of HIV in urine utilizes enzyme-labeled antibodies to one or more of the HIV markers. Techniques for conjugation of enzymes to antibodies are well known in the art. Many different enzymes or co-enzymes can be utilized for this purpose; for example, galactosidase can be conjugated to antibody to anti-HIV (i.e. anti-anti-HIV-Gal). This complex forms the active portion of R-1, and is mixed first with sample. If anti-HIV is present in the sample, it binds to the antibody, thereby causing release of the enzyme, galactosidase. The R-2 containing a substrate-indicator selected to complement the enzyme (e.g. 5-bromo-6-chloro-3-indoxyl-beta-D-galactopyranoside) is then added to the reaction mixture. The free galactosidase then attacks the substrate complex causing release of the color indicator yielding its characteristic color (e.g. magenta). If no anti-HIV is present, the enzyme remains bound to the antibody complex and no color is produced. Consequently, the color produced is proportional to the concentration of anti-HIV present in the sample. Please note this method can be adapted to dry chemistry dipstick technology. First, the solid paper matrix is immersed in the R-2 reagent, and finally in the R-1 reagent. Alternatively, two distinct paper matrices, or test pads could be used (one for each immersion solution), and a “sandwich” made by stacking the two pads, one on top of the other. The paper pad containing the R-1, however, must be on top of the test pad containing R-2. This process can be utilized for any of the above test methods as long as the indicator yields a color response in the visible color spectrum.

The present invention encompasses a method that can utilize several different techniques. The first technique employs a liquid reagent compatible with most chemistry analyzers currently used for clinical chemistry testing to quantitate the amount of HIV antibody antigens or markers are present in each sample. In addition, this liquid reagent can also be used in classical wet chemistry and spectroscopy techniques. The second technique employs the dry chemistry dipstick (DCD) method. A third technique employs a combination of DCD and thin layer chromatography called a lateral flow device (LFD). Utilization of the liquid reagent with the automated chemistry analyzer facilitates high volume testing (i.e. thousands per hour) and permits testing for HIV while simultaneously running routine chemistries on the same sample using the same analyzer. In the case of testing on a spot urine sample, the additional tests would include creatinine, cystatin C and/or other “normalizing” factors such as osmolality, or specific gravity. The current analyzers can also perform the math required to yield a normalized HIV value (e.g. anti-HIV quantitation/creatinine concentration (ratio) or anti-HIV quantitation/cystatin C concentration (ratio)). The resulting report generated includes HIV and HIV ratio results and all the routine chemistry as requested by the physician. This unified report allows the physician to evaluate test results and report findings rapidly and efficiently. It may also facilitate further testing, and/or prevent costly additional tests.

The automated analysis procedure encompasses the following automated method for the measurement of HIV on an unknown sample of urine (or other biological sample including serum, whole blood, cerebral spinal fluid, gastric fluid, sweat extracts hair homogenates, and saliva). A method to determine AIDS by measuring the concentration of anti-HIV or anti-HIV marker or antigen in a test specimen, said test method comprising the steps of placing the reagent composition(s), R-1 and R-2, in the reagent compartment of the chemistry autoanalyzer, aliquoting samples, calibrators, and controls into sample cups and placing them on the chemistry autoanalyzer, transferring an aliquot of each sample, calibrator, and control into single, discrete cuvettes mounted within the chemistry autoanalyzer, aliquoting a specified volume of the first reagent composition, R-1, into each cuvette and mixing, incubating the reaction mixture for a specified time interval, aliquoting a specified volume of the second reagent composition, R-2 (if required), into each cuvette and mixing, incubating the reaction mixture for a specified time interval, measuring and recording absorbance values of the reaction mixtures with the chemistry autoanalyzer's spectrophotometer at specified wavelength (from 340 to 800 nm) and at preprogrammed time intervals, and comparing absorbance values of samples and controls to those of the calibrators in the form of a standard curve thereby quantitating the anti-HIV present.

The other techniques, dry chemistry dipsticks (DCD's), and lateral flow devices (LFD's) are solid phase assays that use an absorbent medium such as paper which has been impregnated with the chemical formulations needed to perform the assay. To summarize more specifically the foregoing dry chemistry test strip (DCD) method for the measurement of the anti-HIV concentration in a urine sample, said test method comprising the steps of preparing a test means by successively impregnating a carrier matrix with reagent solutions, drying said test means, dipping completed test means into test sample, and determining the quantity of anti-HIV in said test sample by comparing the relative intensity of the color produced by the reaction to a color chart with color blocks referenced to specific concentrations of anti-HIV. To summarize more specifically the foregoing lateral flow test device (LFD) method to determine HIV by measurement of the anti-HIV concentration in a urine sample, said test method comprising the steps of preparing a test means by successively impregnating a carrier matrix with reagent solutions at specific target locations on said test means, drying said test means, dipping into or depositing an adequate amount of test sample to the device at the starting point of the analysis, allowing sufficient time to complete the migration of sample to the end point of the analysis, and determining the presence or absence of anti-HIV in said test sample by comparing the lines produced by the reaction to a result chart for concentrations of anti-HIV. Ease of use and rapid results obtained mark the unique utility of these testing techniques. In addition, very little technical expertise is required to perform these types of assays (i.e. DCD's and LFD's).

A thorough search of the literature reveals no relative art resembling this technology; therefore, this invention is clearly a novel creation, and is not obvious to anyone skilled in the art of determination of HIV (anti-HIV in urine or other biological fluids).

### DETAILED DESCRIPTION OF THE INVENTION

The instant invention provides test strips (i.e. DCD's and LFD's) or automated liquid chemistries for the detection of HIV antibodies through the use of several markers or antigens in urine resulting in the concomitant determination of HIV with a hitherto unachievable high level of ease of use and sensitivity. Essentially, the present invention comprises test strips (carrier dependent, solid phase) or liquid reagents (carrier independent, aqueous phase).

The DCD/LFD rapid test strips and the aqueous, liquid chemistry reagents consist of an indicator(s) (calorimetric, enzymatic, fluorescence, turbidimetric, radioimmuno, antibody, ion-exchange, or ionic) that is specific for the HIV antibodies, and a measurable test means that produces a visual, spectrophotometric, turbidimetric, fluorescence, or reflectance result.

One novel aspect of this new art eliminates the need to use the prior art methods of detection, specifically HPLC and ELISA methods, which are tremendously tedious an time consuming. The present art's ability to increases sensitivity and accuracy, with a test means that is applicable to DCD's, LFD's, and aqueous, liquid reagents compatible with automated analyzers is a tremendous advancement in the art that will significantly lower the cost of the testing and improve results. Another important and novel aspect of this new art is its ability to utilize random urine without predilution or pretreatment of the sample. It is well known in the art that all current ELISA techniques require pretreatment of the test sample. The present device has no such requirement.

This new art's ability to analyze test samples using DCD or LFD technology can not be stressed enough. This one important leap in technology allows the physician at his or her office, and the patient at home to test for osteoporosis without a laboratory. This will have a tremendous impact for thousands of AIDS victims by providing an inexpensive and accurate method for early detection of this insidious disease.

The detection methods of the present device constitute the heart of the analytical response provided by it, and is comprised of one or more reagent compositions responsive to HIV antibodies, and produces a detectable response. These test means are thus able to interact with the HIV antibodies in a test sample, and yield a detectable response which enables the interpretation of HIV exposure and possible development of AIDS. The response can be in the form of the appearance or disappearance of a color or line, or the changing of one color to another. Said measurable response may also be evidenced by a change in the amount of light reflected or absorbed during the reaction of interest. The analytical arts are replete with examples of these types of detectable responses. Thus the reagent composition of the present device constitutes the heart of the analytical process, and in the broadest sense includes one or more reagent compositions composed of chemical compounds responsive to the analyte of interest thereby producing some detectable manifestation of the presence of said analyte of interest (i.e. selected bone antigens). The response can be in the form of the appearance, disappearance, or change in intensity of one or more colors in the ultra violet or visible spectrum. Such changes can be measured with a spectrophotometer or colorimeter using direct absorbance or reflectance. In the case of the visible spectrum, the human eye can also determine the color changes or the appearance of a colored line.

Consequently, according to the present invention, there is provided a method for determining HIV exposure by the measurement of HIV antibodies on an unknown test sample or urine, said test method being composed of a buffer and an indicator reagent that produces a color change, or a change in the absorbance or intensity of the color in the UV or visible spectrum in the presence or absence of bone loss markers.

Those skilled in the prior art could not have been foreseen the development of this new art and the tremendous advancement it represents in the diagnosis and treatment of AIDS. It is important to note the present invention can utilize urine specifically, but it may also be equally effective with blood, serum, saliva, and cerebral spinal fluid.

The instant invention is comprised of a reagent containing an enzyme and/or an antibody and/or an indicator, and buffer. Optional components include a substrate, surfactant (i.e. wetting agent), and compounds for removal of interfering substances. A few substances which remove sample matrix interference's include mono, di, tri, and tetra sodium salts of EDTA or EGTA. One or more of these interference-removing compounds can be mixed with the test specimen as part of the R-1 of the reagent composition. Note, this instant invention will be referred to hereafter as HIV reagent. Buffering of the reactants acts to stabilize pH. It is well known in the art that most reactions have an optimum pH range, and an ideal buffer should be selected on that basis. Usable buffers may include the following listed by their common names: citrate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, phosphate, phthalate, and succinate. The proper chemical names for the above buffers and their common counterparts may be found in the Sigma Chemical Catalog, 1999, pages 1910 to 1917. In addition, organic and inorganic acids and bases may also be used in the buffering process and may include hydrochloric, phosphoric, sulfuric, nitric, and acetic acids, and hydroxides such as NaOH and KOH.

In the case of the liquid reagent, the chemical composition is dissolved in water, and the pH of the solution(s) is adjusted. In some circumstances, the analysis may require a two-part reagent system, or two solutions. The analysis proceeds by placing reagent and samples on the automated chemistry analyzer; samples, standards and controls are then pipetted from the sample cups into reaction cuvettes, mixed with reagent which is added to the cuvettes, and absorbance readings (taken at a specified time interval using a preprogrammed wavelength) are taken, stored, and compared to known standard values to quantitate the amount of HIV antibody each unknown.

In the case of DCD technology, the manufacturing process includes impregnating onto an absorbent, solid carrier (e.g. paper) the chemical constituents which have been dissolved in a liquid solvent, evaporating the solvent, and mounting this “reaction paper” on a sturdy plastic “handle”; this device is then dipped into the test sample, withdrawn, and the visible color produced is observed and compared to a chart which relates specific colors or shades of the same color to a range of concentrations of the target analyte. Note the absorbent paper can also act as the support handle.

In the case of LFD technology, the manufacturing process includes impregnating onto an absorbent, solid carrier (e.g. paper) the chemical constituents which have been dissolved in a liquid solvent, evaporating the solvent, and mounting this “reaction paper” on a solid support which can encapsulate the LFD test pad except for the point of application of sample, and any areas in which results (e.g. colors or lines) are to be observed; sample is then placed on the device at the bottom or starting point for the assay, and after the simple has migrated to the top of the test pad, the appearance of lines on the device is compared to the result chart and results are recorded. Note, the test pad must be an absorbent wicking material that permits migration of sample up the solid absorbent test pad and allows analytes and reactants to interact at specific binding sites along the test pad.

The following is a brief explanation of the LFD technology of this invention, and will be described in detail in the following examples. This example is purely illustrative and this art is not limited to this description. This HIV LFD device is approximately 5 mm wide by 70 mm long. The absorbent material is cut to fit these dimensions. For this example the device will use anti-HIV cutoffs of 10.0 fmol/L anti-HIV (the presence of any anti-HIV in urine is considered a positive). The starting point or origin at which the sample is placed on the test device is 5 mm from one end of the strip, and 30 mm from this origin a buffered solution containing anti-anti-HIV is bound to the test strip 35 mm from the bottom edge of said test pad in a line approximately 1 mm wide by 5 mm long thereby extending from one side of the device to the other side forming the C, “control line”. The appearance of a colored line here after assay is complete will indicate that the lateral flow device worked properly (i.e. the sample migrated to an acceptable RF value beyond the A or assay line and the binding capacity of the HIV antigen and the bound anti-anti-HIV are reactive and nothing in the sample has adversely affected the test's reactants). A second buffered solution consisting of blue colored particles bound (i.e. irreversibly coupled, conjugated, or covalently linked) to 100 fmol/L HIV antigen, and 10 fmol/L of blue colored reacted particles bound to anti-HIV-HIV-antigen (control particles, these control particles will not react with the anti-HIV in the urine or the bound HIV antigen because the reactive sites are already occupied) is applied to the strip approximately 5 mm from the starting point (or 10 mm from the lower edge of the test strip) in a concentration as to make certain that assay and control lines both form solid visual lines to achieve effective results. A third buffered solution of anti-HIV is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled anti-HIV line. A solid plastic case may be used to conceal and protect all of the device except for three “windows”; one for sample application at the origin, a second at the A, assay line, and a third at the C, control line.

If the sample is positive, with a concentration of 10.0 fmol/L anti-HIV or more the following occurs. A drop of urine (approximately 50 uL) is applied at the starting point or origin of the strip. The urine then migrates to the opposite or terminal end of the strip. The free anti-HIV present in the urine binds all of the HIV antigen conjugated to the colored particles and these anti-HIV-HIV-antigen blue particle complexes also migrate with the urine toward the terminal end of the strip away from the starting point. These colored complexes will not bind to the line of bound anti-HIV at the 10 mm “A” line or assay window because all of the HIV antigen on the colored particles is already bound up by the free anti-HIV from the sample. The migrating blue colored particle complexes, therefore, continue migrating up the device until reaching the line of bound anti-anti-HIV at the “C” or control window. The anti-HIV-HIV-antigen blue particle complexes then bind to this line of anti-anti-HIV forming a solid (complete) blue “control line” consisting of anti-HIV-HIV-antigen blue particles and the control particles.

If the sample is negative, with a concentration of less than 10 fmol/L of anti-HIV, the following occurs. The free (unbound) HIV antigen blue particle complexes migrate up to the “A” assay line and bind to the immobilized (bound) anti-HIV conjugated to the test strip at that location thereby forming a solid (complete) blue line. The control particles (i.e. free anti-HIV-HIV-antigen blue particle complexes) will keep migrating to the 35 mm “C” control line and form a solid blue line to indicate the assay worked properly.

This brief description of the present art illustrates a completely enabled device that would allow a physician, patient, and/or technician to quickly and easily determine the anti-HIV value or HIV exposure of the patient. The anti-HIV normal value for this example is less than 10 fmol/L anti-HIV. A value of equal to or greater than 10 fmol/L of anti-HIV indicates the patient has been exposed to HIV. If analysis is performed on a 24 hour urine collection, no further analysis is required. Proper 24 hour urine collections are difficult and inconvenient for the patient, however, the above test can also be performed using a random specimen. Consequently, a novel addition to further improve the ease of use and the accuracy of the present device requires an additional assay on the same random or spot urine used for the HIV assay. This additional assay is for creatinine or cystatin C. These analyte values can be used to “normalize” or correct the HIV result for the amount of water present in the sample. Water content of a random urine sample is affected by the diurnal variations, diet, diuretics (e.g. caffeine, sugar) and short term fluid consumption (water consumed over the previous 2 to 3 hours). The amount of creatinine or cystatin C excreted by a normal, healthy individual is relatively consistent from day to day, and hour to hour; any HIV antibodies if present would also be excreted at a consistent rate from hour to hour. Creatinine and Cystatin C are, therefore, ideal for adjusting or normalizing the amount of anti-HIV found in a random urine.

Obviously if the creatinine or cystatin C concentration is high the subject has consumed very little water over the previous few hours, and the anti-HIV value will be elevated; if the subject has consumed a large volume of water just prior to testing, the creatinine or cystatin C value will be low and the anti-HIV concentration will also be depressed.

The following formula may be used to adjust the HIV value according to the creatinine or cystatin C concentration. In this example creatinine will be used instead of cystatin C or some other steady state marker. This example requires multiplication of the marker value by the volume of urine (50 uL in the above example) divided by the creatinine concentration of the sample. This yields a normalized anti-HIV value for a random sample. The method of measuring creatinine in urine by LFD is hitherto unknown in the art until the present device and examples of this methodology will follow. If analysis is being performed via automated chemistry, a number of methods are currently available. And finally this present art incorporates the unique invention of anti-HIV assay with the use of a ratio of anti-HIV to creatinine. This is the value of the anti-HIV divided by the concentration of creatinine. This ratio provides the most convenient way to normalize the anti-HIV value and allow the user, even an untrained one, to obtain a corrected HIV value.

Two additives are typically included in the production of dry chemistry test strips. These are thickening agents and wetting agents. The latter is also an integral part of liquid reagent compositions. The relatively large amount of water-soluble substances present in the recommended formulations tend to promote “bleeding” (i.e. seeping out of the test pad upon re-wetting with test specimen or additional reactants in successive immersions); thickening agents prevent or limit this phenomenon. Some typical compounds used for this purpose include polyvinylpyrrolidone, algin, carrageenin, casein, albumin, methyl cellulose, and gelatin in concentrations ranging from 0.5 to 5 g. per 100 ml. Wetting agents are also typically recommended to aid in even distribution of reactants and even color development. Compounds typically used for this purpose include long chain organic sulphates or sulphonates (e.g. Brij-35, Tween 20, Triton X-100, dioctyl sodium sulphosuccinate, and sodium lauryl sulphate). Wetting agents are typically added to impregnation solutions in amounts from 0.5 to 5 percent. In liquid reagents to be used on automated analyzers, wetting agents improve solubility of reactants, improve flow characteristics through the instrument's tubing, increase distribution and development of color, and reduce formation of bubbles in solution.

Production of the test strips according to the present invention requires an absorbent carrier which may be any of the following: filter paper, cellulose, lateral flow paper/material, and synthetic resin fleeces. Immersion solutions may be aqueous or volatile, organic solvents. The order of application and number of immersion solutions will vary according to the specific assay reaction to be utilized (see examples in this section).

The following are examples of groups of indicator compounds that will function in dry and liquid chemistry anti-HIV urine assays:

I. Indicators

1. Color indicators which produce color by oxidation/reduction

2. uv-visible color indicator bound to Enzyme-specific substrate

3. Enzymatic indicator

4. Fluorescence indicator

5. Turbidimetric indicator composed of aggregate-forming microparticles

6. Ionic indicator

7. uv and visible indicators bound to specific antigen to analyte of interest.

8. uv and visible indicators bound to specific antibody to analyte of interest.

9. Antibodies and Antigens that react with a anti-HIV.

Consequently, according to the present invention, an assay means for the determination of exposure to the HIV via measurement of HIV antibodies in urine, or other biological specimens, may comprise either a test strip composed of a solid, carrier matrix in the form of absorbent paper impregnated with a reaction mixture containing an indicator compound of the general formula (I), dried, and attached to a sturdy handle to form a dry chemistry dipstick (DCD) or lateral flow device (LFD), or a liquid reagent composed of an aqueous solution containing an indicator compound of the general formula (I) that is compatible with most general chemistry auto-analyzers.

Development of the present invention and the concomitant extraordinary increase in utility of it is not obvious in view of the prior art. The present invention targets urine in particular, but other biological fluids are well within the scope of this novel technology including saliva, gastric juices, cerebral spinal fluid, blood, serum, sweat, and hair extracts.

The following examples are provided to further illustrate the inventive aspects of the present discovery, and to further describe preferred embodiments. As such, they are intended as being merely illustrative, and are not to be construed as limiting the scope of the claims appended hereto.

The first calorimetric antibody method utilizes the direct interaction between colored particle bound to an HIV antigen and anti-anti-HIV in the presence of any free anti-HIV in the test sample as previously described.

### EXAMPLE 1

The following procedure is a method for manufacturing a dry chemistry, lateral flow test strip for the determination of HIV viral exposure by measurement of a sample's anti-HIV concentration; in this example the targeted is anti-HIV. This example will also illustrate the utility of incorporating the use of creatinine concentration (as determined by calorimetric assay, DCD, LFD, antibody/antigen, etc . . . ) on the same sample measured for HIV and the enhanced clinical significance of the anti-HIV value.

Absorbent material is successively impregnated with the following solutions and dried at 25 degree C.:

Solution 1

0.05 M Phosphate buffer pH 7.2

100 fmol/L anti-IgG

Solution 2

0.05 M Phosphate buffer pH 7.2

30 fmol/L HIV antigen conjugated to red microparticles

30 fmol/L IgG conjugated to red microparticles

Solution 3

0.05 M Phosphate pH 7.2

30 fmol/L anti-HIV

In this example, the lateral flow device is prepared in accordance with the instant invention. The lateral flow device is comprised of a paper carrier matrix impregnated with the compositions of solutions 1, 2, and 3 above. Note that said concentrations of any of the above constituents can be varied to suit the lateral flow/dipstick device format (e.g. dependent upon paper type, and inclusion of semi-permeable membranes or other innovations utilized in dry chemistry technology). Production of this test device is carried out using the following procedure. The test device made up of a solid support which includes an absorbent material capable of transporting a liquid by capillary action or wicking (e.g. nitrocellulose 5.0u, S&S brand) in this example having dimensions of 5 mm by 70 mm and can be backed by or in contact with strips of glass fiber (e.g. Whatman GF/A) to aid in controlling the wicking action. In this example, the device uses an HIV cutoff of 10 fmol/L anti-HIV.

The starting point or origin at which the sample is placed on the test device is 5 mm from one end of the strip, and 30 mm from this origin a buffered solution containing anti-IgG is bound to the test strip 35 mm from the bottom edge of said test pad in a line approximately 1 mm wide by 5 mm long thereby extending from one side of the device to the other side forming the C, “control line”. The appearance of a colored line here after assay is complete will indicate that the lateral flow device worked properly (i.e. the sample migrated to an acceptable RF value beyond the A or assay line and the binding capacity of the anti-HIV and the bound anti-HIV are reactive and nothing in the sample has adversely affected the test's reactants). A second buffered solution consisting of red colored particles bound (i.e. irreversibly coupled, conjugated, or covalently linked) to 30 fmol/L of HIV antigen and 30 fmol/L of anti-IgG (control particles) is applied to the strip approximately 5 mm from the starting point (or 10 mm from the lower edge of the test strip) in a concentration as to make certain that assay and control lines both form solid visual lines to achieve effective results. A third buffered solution of anti-HIV is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled anti-HIV line. A solid case may be used to conceal and protect all of the device except for three “windows”; one for sample application at the origin, a second at the A, assay line, and a third at the C, control line. This case may be composed of plastic, wood, cardboard, or other suitable material.

If the sample is positive, with a concentration of 10 fmol/L anti-HIV or more the following occurs. A drop of urine (approximately 50 uL) is applied at the starting point or origin of the strip. The urine then migrates to the opposite or terminal end of the strip. The free anti-HIV present (in a concentration of 10 fmol/L or greater of anti-HIV) in the urine binds all of the red particles bound with the HIV antigen (10 fmol/L) and these anti-HIV-HIV-antigen red particles complexes will migrate with the urine toward the terminal end of the strip away from the starting point. These colored complexes will not bind to the line of anti-HIV bound at the 10 mm “A” line or assay window because all of the HIV antigen bound colored particles are already bound up by the free anti-HIV from the sample. The migrating red colored particle complexes, therefore, continue migrating up the device until reaching the line of bound anti-IgG at the “C” or control window. The anti-HIV-HIV-antigen red particle complexes and the anti-IgG red colored particles then bind to this line of anti-IgG forming a solid (complete) red “control line” consisting of both types of red particle complexes.

If the sample is negative, with a concentration of less than 10 fmol/L of anti-HIV is present, the following occurs. The free (unbound) HIV antigen red particles complexes migrate up to the “A” assay line and bind to the anti-HIV conjugated to the test strip at that location thereby forming a solid (complete) red line assay line. The control particles (i.e. IgG red particle complexes) will keep migrating to the 35 mm “C” control line and form a solid red line to indicate the assay worked properly.

The test strip can be placed on top of, or backed, with glass fiber (e.g. Whatman GF/A) in order to control (i.e. speed up, or slow down the “wicking” speed) and held in place by an adhesive or other means. This brief description of the present art illustrates a completely enabled device that would allow a physician, patient, and/or technician to determine rapidly the presence or absence of anti-HIV in a patient's urine. The normal value for this HIV assay is less than 10 fmol/L of anti-HIV detected (i.e., no anti-HIV present in the urine).

If analysis is performed on a 24 hour urine collection, no further analysis is required. Proper 24 hour urine collections are difficult and inconvenient for the patient, however, the above test can also be performed using a random specimen. Consequently, a novel addition to further improve the ease of use and the accuracy of the present device requires an additional assay on the same random or spot urine used for the HIV assay. This additional assay is for creatinine, cystatin C or any other steady state marker consistently excreted in human urine. This analyte value can be used to “normalize” or correct the HIV test result for the amount of water present in the sample. Water content of a random urine sample is affected by the diurnal variations, diet, diuretics (e.g. caffeine, sugar, etc . . . ) and short term fluid consumption (water consumed over the previous 2 to 3 hours). The amount of creatinine excreted by a normal, healthy individual is relatively consistent from day to day, and hour to hour; any anti-HIV would also be excreted at a consistent rate from hour to hour. Creatinine or Cystatin C is, therefore, ideal for adjusting or normalizing the amount of anti-HIV found in a random urine. Specifically, if for example the creatinine concentration is high the subject has consumed very little water over the previous few hours, and the anti-HIV value will be elevated; if the subject has consumed a large volume of water just prior to testing, the creatinine value will be low and the anti-HIV marker will also be depressed.

This present art incorporates the unique invention of the anti-HIV, or anti-HIV steady state marker such as creatinine or Cystatin C ratio (anti-HIV/creatinine). The following formula may be used to adjust the anti-HIV value according to the creatinine concentration, and thereby produce the anti-HIV/creatinine ratio (i.e. H/C ratio). This method requires division of the anti-HIV value by the creatinine concentration of the sample. This yields a normalized anti-HIV value for a random sample. The method of measuring creatinine in urine by LFD is hitherto unknown in the art until the present device and examples of this methodology will follow. If analysis is being performed via automated chemistry, a number of well known methods are currently available. This ratio provides the most convenient way to normalize the anti-HIV value and allow the user, even an untrained one, to obtain a corrected anti-HIV value.

The following is a detailed description of how the HIV/creatinine ratio is used. Obviously, in the case of testing the sample with aqueous, liquid reagents on an automated chemistry analyzer system quantitative results would be obtained for both analytes. The anti-HIV value is then divided by the creatinine concentration. If this ratio is equal to, or greater than 0.054, then exposure to the HIV virus has occurred and appropriate treatment should be initiated. Values lower than 0.054 are considered negative for anti-HIV for this example.

In the example above, the device detects 10 fmol/L of anti-HIV or more in the urine, so positives are considered 10, and negatives are zero. Typical creatinine values range from 45 to 180 mg/dl. Therefore, if the anti-HIV result is positive and the creatinine value is less than 185 mg/dl, then the corrected result is still positive (10/185=0.054); the ratio is inversely proportional to the creatinine value (i.e. as the creatinine drops, the ratio increases). Obviously the higher the ratio, the more HIV exposure. Therefore, a semi-quantitative anti-HIV/creatinine ratio can be obtained by assuming any positive is 10 fmol of anti-HIV and dividing it by the creatinine quantitation (e.g. 10/60=0.166 ratio). On the other hand, if the creatinine concentration is higher than 185, then the true anti-HIV value may be falsely elevated, and a new sample should be tested because this could be interpreted as a false positive.

Conversely, if the osteoporosis value is negative, and the creatinine value is 157 mg/dl or higher, then the sample is clearly negative (5 fmol/L/157 mg/dl=0.0.031). On the other hand if the creatinine value is lower than 20 mg/dl creatinine the assay should be repeated. It is well known in the art that a creatinine of less than 20 mg/dl is a dilute specimen and a false negative could occur with this specimen (5/20=0.25, a positive).

Another factor that can and should be taken into account is kidney function as determined by the protein/creatinine ratio. If the protein/creatinine ratio is normal (less than 3.0, as known in the art), then the assay is not affected by the ability of the kidneys to clear creatinine or other steady state marker such as cystatin C and allow for an accurate assessment of the urine concentration. If the protein/creatinine ratio is greater than 3.0, then the assay can be affected by the kidney function. The anti-HIV/creatinine ratio may be corrected for kidney dysfunction by dividing it by the protein/creatinine ratio (i.e. H/K ratio), and determining appropriate ranges. Preliminary data suggests that an H/K ratio of 0.05 or higher is normal, and an H/K ratio of less than 0.05 indicates bone loss.

To summarize Example 1 more specifically, the foregoing lateral flow/dry chemistry test strip (LFD) method for measuring the anti-HIV concentration for the determination of exposure to the HIV virus and possibly AIDS in a random urine sample, the method comprising the steps of preparing a test means by successively impregnating a solid, absorbent, carrier matrix with liquid reagent solutions at specific locations on said test means, drying said test means, dipping completed test means into test sample or pipetting sample onto the test means, and determining the quantity of anti-HIV in said test sample by comparing the relative intensity (completeness) of the assay line produced by the reaction to the control line. Also, the assay can include the determination of creatinine to determine the anti-HIV/creatinine ratio (H/C ratio) to improve the validity of the test result. It is understood that the above example was purely illustrative, and that the relative positions of the control and assay lines could be relocated without changing the spirit, scope, or intent of the instant invention.

Changes to the foregoing solutions could be made and still have similar results. The foregoing solutions could be combined together, or reduced to include only 1 solution for impregnation. The concentrations of said constituents may also be changed and still remain within the scope of the invention. The antibody to HIV can be to HIV 1 or HIV 2 virus in this example the antibodies can be replaced with antigens in appropriate positions to make for a different format than explained in the example. Anti-Anti-HIV could be used which is the antibody to the HIV antibody. The foregoing was merely illustrative of the possibilities of this novel and unique invention.

A thorough search of patents and published research has revealed no relative art (i.e., prior art) even slightly resembling this technology. Other than the art of manual methods (ELISA and HPLC) described above, no similar chemical test means has been described prior to the disclosure of this method.

As taught and can be substituted for the reactants that target and react with the HIV antibody as shown in EXAMPLE 1 are the following anti-anti-HIV (I or II), anti-HIV (I or II), HIV antigens (I or II), anti-IgG, anti-IgM or other human antibodies or HIV aptamers. All of these reactants can be used and will produce a detectable response in the presence of HIV antibody.

The buffers used in example 1, may be substituted with any one or more from the following list: citrate, phosphate, phthalate, acetate, hydrochloric acid, oxalate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, and succinate. Note: the brand names, trade names, common names, and abbreviations above are commonly used and can be found in the 1999 SIGMA Chemical catalog page 1910.

The colored particles used in example 1 could be replaced with particles of any color, and made from many types of materials including rubber, latex, plastics, synthetic solids, metals, or other suitable material that will form a solid platform or substrate for the covalent attachment (binding) of a reactive compound, antibody, and/or antigen to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

### EXAMPLE 2

The following procedure is a method for manufacturing a lateral flow device (LFD) for determining the concentration of an anti-HIV, and cystatin C simultaneously without the aid of any other instrumentation. This lateral flow device will hold one LFD strip for anti-HIV, and one LFD strip for cystatin C in two separate channels. The lateral flow device may have the following dimensions, but can obviously be changed and still remain within the spirit and scope of the present invention. This device is approximately 100 mm long by 50 mm wide. The device is approximately 3 to 5 mm thick. The two absorbent test pads are 5 mm wide and 70 mm long. The two channels will have two holes or windows each. Each assay channel has one assay hole through which the reaction and assay results can be observed; these viewing windows are 50 mm long by 5 mm wide. Each assay channel also has two window through which to introduce sample onto the test pads. These sample holes or ports are approximately 10 mm long by 3 to 5 mm wide. The two assay channels may be on the same side of the device or one on each of the two sides. Note, the assay and sample windows are aligned with the appropriate areas of the test strips so that sample is applied to the correct location, and the appropriate reaction areas are open to view. The casing is composed of plastic, rubber, latex, wood, cardboard, or other suitable material.

The anti-HIV assay strip is identical to the one described in Example 1, and is placed in channel 1. The second strip is made according to the following and will be placed into channel two:

An absorbent material is successively impregnated with the following solution and dried at 25 degree C.:

Solution 1

0.05 M Tris buffer pH 7.2

150 mg/dL anti-IgG

Solution 2

0.05 M Tris buffer pH 7.2

150 mg/dL IgG conjugated to blue micro-particles

150 mg/dL anti-cystatin C conjugated to blue micro-particles

Solution 3

0.05 M Tris buffer pH 7.2

150 mg/dL cystatin C

In this example, the lateral flow device is prepared in accordance with the instant invention. The lateral flow device is comprised of a absorbent paper carrier matrix impregnated with the composition of solutions 1, 2 and 3 from above in the appropriate locales as specified below. Note that said concentrations of any of the above constituents can be varied to suit variations employed in the specific LFD format (e.g. particular paper type, or inclusion of semi-permeable membranes or other innovations utilized in dry chemistry technology). Production of this test device is carried out using the following procedure. The test device made up of a solid support which includes an absorbent material capable of transporting a liquid by capillary action (wicking) on a piece of filter paper (e.g. nitrocellulose 5.0u, S&S brand) in this example having dimensions of 5 mm by 70 mm and can be backed by or in contact with strips of glass fiber (e.g. Whatman GF/A) to aid in controlling the wicking action. In this example, the device for illustration purposes uses a Cystatin C concentration cutoff of 150 mg/dL.

The starting point or origin at which the sample is placed on the test device is 5 mm from one end of the test pad, and 30 mm from this origin buffered solution no. 1 containing anti-IgG is permanently bound to the test strip 35 mm from the bottom edge of said test pad in a line approximately 1 mm wide by 5 mm long thereby extending from one side of the device to the other side forming the C, or “control line”. The appearance of a colored line here after assay is complete will indicate that the lateral flow device worked properly (i.e. the sample migrated to an acceptable RF value beyond the A or assay line and the binding capacity of the anti-cystatin C and the bound cystatin C are reactive and nothing in the sample has adversely affected the test's reactants). The buffered solution no. 2 consisting of blue colored particles bound (i.e. irreversibly coupled, conjugated, or covalently linked) to 150 mg/dL anti-Cystatin C, and 150 mg/dL of blue colored particles bound to IgG (control particles) is applied to the strip approximately 5 mm from the starting point (or 10 mm from the lower edge of the test strip) in a concentration as to make certain that assay and control lines both form solid visual lines to achieve effective results. The third buffered solution, no. 3, of 150 mg/dL cystatin C is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled creatinine line. The test strips can be placed on top of, or backed, with glass fiber (e.g. Whatman GF/A) in order to control (i.e. speed up, or slow down the “wicking” speed) and held in place by an adhesive or other means. The two assay strips are then placed in the solid case to conceal and protect all of the device except for the two “windows”; one for sample application at the origin, and the second to display the A, assay line and the C, control line.

The HIV test channel is read and evaluated as described in Example 1. The cystatin C test channel is interpreted as follows. If the sample has an abnormally high concentration of 150 mg/dL cystatin C or more the following occurs. A drop of urine (approximately 50 uL) is applied at the starting point or origin of the strip. The urine then migrates to the opposite or terminal end of the strip. The free cystatin C present in the urine binds all of the anti-cystatin C conjugated to the colored particles and these cystatin C/anti-cystatin C/blue particle complexes also migrate with the urine toward the terminal end of the strip away from the starting point. These colored complexes will not bind to the line of cystatin C bound at the 10 mm “A” line in the assay window because all of the anti-cystatin C on the colored particles is already bound up by the free cystatin C from the sample. The migrating blue colored particle complexes, therefore, continue migrating up the device until reaching the line of bound anti-IgG at the “C” line in the assay window. The cystatin C/anti-cystatin C/blue particle complexes then bind to this line of anti-IgG forming a solid (complete) blue “control line” consisting of cystatin C/anti-cystatin C/blue particle/anti-IgG complexes.

If the sample is normal, with a concentration of less than 150 mg/dL, the following occurs. The free anti-cystatin C/blue particle complexes migrate up to the “A” assay line and bind to the cystatin C conjugated to the test strip at that location thereby forming a solid (complete) blue line. The control particles (i.e. free cystatin C/anti-cystatin C/blue particle complexes) will keep migrating to the 35 mm “C” control line and form a solid blue line to indicate the assay worked properly.

This brief description of the present art illustrates a completely enabled device that would allow a physician, patient, and/or technician to determine rapidly the cystatin C value of the patient. In this assay's example normal value for cystatin C merely for illustrative purposes is 45 to 180 mg/dL; the utility of this assay in conjunction with the anti-HIV assay will be explained herein. As described in Example 1, a positive value for the anti-HIV test coupled with a cystatin C value below 150 mg/dL is indicative of a positive result for anti-HIV and suggest that the individual has been exposed to the HIV virus. On the other hand, a positive anti-HIV value coupled with a cystatin C above 150 mg/dL may be falsely elevated. Another specimen should be collected, and tested. It is clear, that this novel assay pair will yield a tremendously valuable diagnostic tool in the universal fight to create a healthier world. Individually each assay is a significant advance in the art of medical diagnosis. In combination, they provide an exponential jump in diagnosis of HIV.

To summarize Example 2 more specifically, the foregoing lateral flow, dry chemistry test strip (LFD) method measures anti-HIV and cystatin C concentration to determine if an individual has had HIV viral exposure by assaying a random urine sample simultaneously without the use of an instrument of other device. This is a marked advance in the art. The method is comprised of the steps of preparing a test means by successively impregnating a solid absorbent carrier matrix with liquid, reagent solutions at specific locations on the test means, drying said test means, applying test sample onto the test device, and determining the quantity of anti-HIV or the cystatin C concentration in said test sample by comparing the relative intensity (completeness) of the assay line produced by the reaction to the control line.

Changes to the foregoing solutions could be made and still have similar results. The foregoing solutions could be combined together, or reduced to include only 1 solution for impregnation. The concentrations of said constituents may also be changed and still remain within the scope of the invention. The cystatin C could be replaced with other cystatin C reactive indicators and still remain within the spirit and scope of the invention. Thus, the cystatin C method taught herein may be replaced with a colorimetric procedure, dry chemistry dipstick, enzyme/substrate assay, or other assay technique.

Currently through patent and research searches reveal no relative art (i.e., prior art) even slightly resembling this technology. Other than the mentioned art of manual methods and other antiquated arts. No chemical test means has been described prior to this art for this method.

The lateral flow strips could be made and impregnated on the same strip instead of using two separate strips. The only difference would be to have just one single control line with the bound anti-IgG and two assay lines, one for anti-HIV and one for cystatin C.

The buffers used in example 2, may be substituted with one or more from the following: citrate, phosphate, phthalate, acetate, hydrochloric acid, oxalate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, and succinate. Note: the brand names, trade names, common names, and abbreviations above are commonly used and can be found in the 1999 SIGMA Chemical catalog page 1910.

The colored microparticles used in example 2 could be replaced with microparticles that have other colors, or composed of rubber, latex, plastic, synthetic solids, metals, or other suitable materials that will form a solid platform, or substrate for the covalent attachment (binding) of a reactive particle, antibody, and/or antigen to it.

As taught and can be substituted for, are the reactants that target and react with creatinine, cystatin C, or other renal markers such as anti-cystatin C, anti-creatinine, anti-IgG, anti-IgM, or other human antibodies. All of these reactants can be used and will produce a detectable response in the presence of renal clearance marker that the reactant is specific for.

### EXAMPLE 3

The following procedure is a method for manufacturing a dry chemistry lateral flow test strip (LFD) for the determination of the presence of anti-HIV in a test sample by measurement for anti-HIV, in this example the target is anti-HIV. This example also illustrates the unique ability to use creatinine or cystatin C measurement on the same sample measured for anti-HIV by assaying for creatinine or cystatin or some other urine clearance marker via liquid colorimetric methodology, dry chemistry (DCD), lateral flow (LFD), liquid antibody/antigen, liquid enzymatic assay, or other techniques to enhance the clinical significance of the he presence of anti-HIV assay value.

Absorbent material is successively impregnated with the following solution and dried at 25 degree C.:

Solution 1

0.05 M Tris buffer pH 7.2

10 fmol/L anti-IgG

Solution 2

0.05 M Tris buffer pH 7.2

100 fmol/L IgG conjugated to colored micro-particles (green)

In this example the lateral flow device is prepared in accordance with the instant invention. This LFD is comprised of a paper carrier matrix impregnated in specific locations on the device with solutions 1 and 2 above. Note, the concentrations of any of the above constituents can be varied to suit the lateral flow/dipstick device format (e.g. dependent on paper type, and inclusion of semi-permeable membranes or other innovations in dry chemistry technology); the specific locations of the solutions may also be varied and still remain within the spirit and scope of this invention.

The test device made up of a solid support which includes an absorbent material capable of transporting a liquid by capillary action or wicking (e.g. nitrocellulose 5.0u, S&S brand) in this example having dimensions of 5 mm by 70 mm and can be backed by or in contact with strips of glass fiber (e.g. Whatman GF/A) to aid in controlling the wicking action. In this example, the device uses an osteoporosis cutoff of 10 fmol/L anti-HIV.

Production of this test device is carried out using the following procedure. The test device made up of a solid support which includes an absorbent material capable of transporting a liquid by capillary action (wicking) on a piece of filter paper (for example, nitrocellulose 5.0u, S&S brand) in this example having dimensions of 5 mm by 70 mm and can be backed by or in contact with strips of glass fiber (for example; Whatman GF/A) to aid in controlling the wicking action. In this example the device uses anti-HIV cutoffs of be 10 fmol/L anti-HIV.

The starting point or origin at which the sample is placed on the test device is 5 mm from one end of the strip, and 35 mm from this origin the buffered solution no. 1 containing anti-IgG is irreversibly bound to the test strip 40 mm from the bottom edge of said test pad in a line approximately 1 mm wide by 5 mm long thereby extending from one side of the device to the other side forming the A, “assay line” and solution no. 1 is also applied to the test strip at the 45 mm mark forming the C, “control line”. This location will indicate the concentration of target analyte present in the unknown sample tested. The second buffered solution consisting of green colored microparticles bound (i.e. irreversibly coupled, conjugated, or covalently linked) to 100 fmol/L IgG is applied to the strip approximately 5 mm from the starting point (or 10 mm from the lower edge of the test strip) in a concentration as to make certain that assay and control lines both form solid visual lines to achieve effective results. A solid case may be used to conceal and protect all of the device except for two “windows”; one for sample application at the origin, and a second at the A, assay line. This case may be composed of plastic, wood, cardboard, or other suitable material.

If the sample is positive (i.e. anti-HIV is present), with a concentration of 10 fmol/L anti-HIV or more the following occurs. A drop of urine (approximately 50 uL) is applied at the starting point or origin of the strip. The urine then migrates to the opposite or terminal end of the strip. The free anti-HIV present in the urine binds all of the anti-IgG bound to the device at the 40 mm mark (i.e. the “A” line; the end result is no solid green colored line will form there. This occurs because the anti-HIV is much smaller than the free IgG microparticles that were impregnated at the 5 mm mark, and therefore migrates to the “A” line faster.

If the sample is negative (i.e. normal, no anti-HIV is present), with a concentration of less than 10 fmol/L anti-HIV, the following occurs. The free IgG green colored particle complexes migrate up to the “A” assay line and bind to the anti-IgG sites conjugated to the test strip at that location thereby forming a solid (complete) green line. This occurs, because there is no free anti-HIV in the sample to bind the anti-IgG sites on the “A” line thereby allowing sufficient numbers of the green IgG microparticles to bind there and form a visible line.

The test strip can be placed on top of, or backed, with glass fiber (e.g. Whatman GF/A) in order to control (i.e. speed up, or slow down the “wicking” speed) and held in place by an adhesive or other means. This brief description of the present art illustrates a completely enabled device that would allow a physician, patient, and/or technician to determine rapidly determine whether a patient has been exposed to the AIDS virus. The normal or negative value for the presence of anti-HIV is less than 10 fmol/L, the abnormal, or positive value is 10 fmol/L or greater.

To further improve the accuracy of the present device the user should perform a creatinine on the sample of urine. This can be accomplished via standard methods currently available (i.e. Jaffe or other colorimetric methodology, or enzymatic assays utilizing automated chemistry analyzers). Alternatively, one may utilize one of the dry chemistry, lateral flow, or antibody/antigen techniques taught herein. This will eliminate diagnostic errors caused by the varying water content in random urine samples, and permit “normalization” or correction of the anti-HIV value. See Example 1 for further elaboration.

To summarize Example 3 more specifically, the foregoing lateral flow/dry chemistry test strip (LFD) method to measure for the presence of anti-HIV in a random urine sample, the method comprising the steps of preparing a test means by successively impregnating a solid, absorbent, carrier matrix with liquid reagent solutions at specific locations on the device, drying said test means, dipping completed test means into test sample or pipetting a known volume of urine onto the test device and determining the quantity of anti-HIV in said test sample by comparing the relative intensity (completeness) of the assay line produced by the reaction to a standard chart or by direct observation. Also, the assay can include the determination of creatinine to calculate the H/C ratio to improve the validity of the test result. It is understood that the above example was purely illustrative, and that the relative position of the assay line could be relocated without affecting the performance of the device, or altering the scope of the invention.

Changes to the foregoing solutions could be made and still have similar results. The foregoing solutions could be combined together, or reduced to include only 1 solution for impregnation. The concentrations of said constituents may also be changed and still remain within the scope of the invention. There can be substitutes other than illustrated in the examples for buffers, antibodies, colored microparticles or other constituents as delineated in Example 1 may also be used for the formulations as outlined for this example and would produce similar results, and still remain within the spirit and scope of the present invention.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffers used in this example may be substituted with any one or more from the following list: citrate, phosphate, phthalate, acetate, hydrochloric acid, oxalate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, and succinate. Note: the brand names, trade names, common names, and abbreviations above are commonly used and can be found in the 1999 SIGMA Chemical catalog page 1910.

The antibodies used in this example and the prior examples may be substituted with any one or more of the following anti-HIV (I or II), HIV antigens (I or II), anti-IgG, anti-IgM or other human antibodies or HIV aptamers. All of these reactants can be used and will produce a detectable response in the presence of HIV antibody.

The colored particles used in this example could be replaced with particles of any color, and made from many types of materials including rubber, latex, plastics, synthetic solids, metals, or other suitable material that will form a solid platform or substrate for the covalent attachment (binding) of a reactive compound, antibody, and/or antigen to it.

### EXAMPLE 4

The following procedure is a method for manufacturing a dry chemistry lateral flow test strip (LFD) for the determination of anti-HIV in a test sample by measurement of anti-HIV concentration, in this example the target is anti-HIV. This example substitutes gold microparticles (metallic) for the microspheres utilized in the previous examples. A buffer appropriate to this material is also substituted.

Absorbent material is successively impregnated with the following solution and dried at 25 degree C.:

Solution 1

0.05 M Hepes buffer pH 7.2

10 fmol/L anti-IgG

Solution 2

0.05 M Mops buffer pH 7.2

100 fmol/L IgG gold particles

In this example the lateral flow device is prepared in accordance with the instant invention. This LFD is comprised of a paper carrier matrix impregnated in specific locations on the device with solutions 1 and 2 above. Note, the concentrations of any of the above constituents can be varied to suit the lateral flow/dipstick device format (e.g. dependent on paper type, and inclusion of semi-permeable membranes or other innovations in dry chemistry technology); the specific locations of the solutions may also be varied and still remain within the spirit and scope of this invention.

The test device made up of a solid support which includes an absorbent material capable of transporting a liquid by capillary action or wicking (e.g. nitrocellulose 5.0u, S&S brand) in this example having dimensions of 5 mm by 70 mm and can be backed by or in contact with strips of glass fiber (e.g. Whatman GF/A) to aid in controlling the wicking action. In this example, the device uses an osteoporosis cutoff of 10 fmol/L anti-HIV.

Production of this test device is carried out using the following procedure. The test device made up of a solid support which includes an absorbent material capable of transporting a liquid by capillary action (wicking) on a piece of filter paper (for example, nitrocellulose 5.0u, S&S brand) in this example having dimensions of 5 mm by 70 mm and can be backed by or in contact with strips of glass fiber (for example; Whatman GF/A) to aid in controlling the wicking action. In this example the device uses anti-HIV cutoffs of be 10 fmol/L anti-HIV.

The starting point or origin at which the sample is placed on the test device is 5 mm from one end of the strip, and 35 mm from this origin the buffered solution no. 1 containing anti-IgG is irreversibly bound to the test strip 40 mm from the bottom edge of said test pad in a line approximately 1 mm wide by 5 mm long thereby extending from one side of the device to the other side forming the A, “assay line” and solution no. 1 is also applied to the test strip at the 45 mm mark forming the C, “control line”. This location will indicate the concentration of target analyte present in the unknown sample tested. The second buffered solution consisting of gold microparticles bound (i.e. irreversibly coupled, conjugated, or covalently linked) to 100 fmol/L IgG is applied to the strip approximately 5 mm from the starting point (or 10 mm from the lower edge of the test strip) in a concentration as to make certain that assay and control lines both form solid visual lines to achieve effective results. A solid case may be used to conceal and protect all of the device except for two “windows”; one for sample application at the origin, and a second at the A, assay line. This case may be composed of plastic, wood, cardboard, or other suitable material.

If the sample is positive (i.e. anti-HIV is present), with a concentration of 10 fmol/L anti-HIV or more the following occurs. A drop of urine (approximately 50 uL) is applied at the starting point or origin of the strip. The urine then migrates to the opposite or terminal end of the strip. The free anti-HIV present in the urine binds all of the anti-IgG bound to the device at the 40 mm mark (i.e. the “A” line; the end result is no solid gold (actually reddish looking) line will form there. This occurs because the anti-HIV is much smaller than the free IgG microparticles that were impregnated at the 5 mm mark, and therefore migrates to the “A” line faster.

If the sample is negative (i.e. normal, no anti-HIV is present), with a concentration of less than 10 fmol/L anti-HIV, the following occurs. The free IgG gold particle complexes migrate up to the “A” assay line and bind to the anti-IgG sites conjugated to the test strip at that location thereby forming a solid (complete) gold line. This occurs, because there is no free anti-HIV in the sample to bind the anti-IgG sites on the “A” line thereby allowing sufficient numbers of the gold IgG microparticles to bind there and form a visible line.

The test strip can be placed on top of, or backed, with glass fiber (e.g. Whatman GF/A) in order to control (i.e. speed up, or slow down the “wicking” speed) and held in place by an adhesive or other means. This brief description of the present art illustrates a completely enabled device that would allow a physician, patient, and/or technician to determine rapidly determine whether a patient has been exposed to the AIDS virus. The normal or negative value for the presence of anti-HIV is less than 10 fmol/L; the abnormal, or positive value is 10 fmol/L or greater.

To further improve the accuracy of the present device the user should perform a creatinine on the sample of urine. This can be accomplished via standard methods currently available (i.e. Jaffe or other calorimetric methodology, or enzymatic assays utilizing automated chemistry analyzers). Alternatively, one may utilize one of the dry chemistry, lateral flow, or antibody/antigen techniques taught herein. This will eliminate diagnostic errors caused by the varying water content in random urine samples, and permit “normalization” or correction of the anti-HIV value. See Example 1 for further elaboration.

To summarize Example 3 more specifically, the foregoing lateral flow/dry chemistry test strip (LFD) method to measure for the presence of anti-HIV in a random urine sample, the method comprising the steps of preparing a test means by successively impregnating a solid, absorbent, carrier matrix with liquid reagent solutions at specific locations on the device, drying said test means, dipping completed test means into test sample or pipetting a known volume of urine onto the test device and determining the quantity of anti-HIV in said test sample by comparing the relative intensity (completeness) of the assay line produced by the reaction to a standard chart or by direct observation. Also, the assay can include the determination of creatinine to calculate the H/C ratio to improve the validity of the test result. It is understood that the above example was purely illustrative, and that the relative position of the assay line could be relocated without affecting the performance of the device, or altering the scope of the invention.

Changes to the foregoing solutions could be made and still have similar results. The foregoing solutions could be combined together, or reduced to include only 1 solution for impregnation. The concentrations of said constituents may also be changed and still remain within the scope of the invention. There can be substitutes other than illustrated in the examples for buffers, antibodies, colored microparticles, gold particles (or other metal particles) or other constituents as delineated in Example 1 may also be used for the formulations as outlined for this example and would produce similar results, and still remain within the spirit and scope of the present invention.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffers used in this example may be substituted with any one or more from the following list: citrate, phosphate, phthalate, acetate, hydrochloric acid, oxalate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, and succinate. Note: the brand names, trade names, common names, and abbreviations above are commonly used and can be found in the 1999 SIGMA Chemical catalog page 1910.

The antibodies used in this example and the prior examples may be substituted with any one or more of the following anti-HIV (I or II), HIV antigens (I or II), anti-IgG, anti-IgM or other human antibodies or HIV aptamers. All of these reactants can be used and will produce a detectable response in the presence of HIV antibody.

The colored particles used in this example could be replaced with particles of any color, and made from many types of materials including rubber, latex, plastics, synthetic solids, metals, or other suitable material that will form a solid platform or substrate for the covalent attachment (binding) of a reactive compound, antibody, and/or antigen to it. Lateral flow test strip (LFD) for the determination of anti-HIV in a test sample by measurement of anti-HIV concentration, in this example the target is anti-HIV. This example substitutes gold microparticles (metallic) for the microspheres utilized in the previous examples. A buffer appropriate to this material is also substituted.

### EXAMPLE 5

A dry chemistry, dipstick (DCD) method for measuring the concentration of creatinine in a random urine sample and used to normalize a diagnostic value (e.g. osteoporosis antigen) obtained on the same sample of urine. This test means includes a buffer and one or more indicators from the following list: an antibody to creatinine, an enzyme specific for creatinine, any pH-sensitive compound, or compound which produces a color or absorbance change in the visible or UV range after undergoing oxidation or reduction. The following example uses a reagent composition of 3,5-Dinitrobenzoic acid, a strong base, and a buffer. In this example, the buffer may include any one or more organic or inorganic acids or bases such as hydrochloric acid, sulfuric acid, nitric acid, borate, phthalate, phosphate, acetic acid, and salts of hydroxides such as NaOH and KOH. The principle: creatinine+3,5-Dinitrobenzoic acid (DNBA)+KOH ref Indigo dye (brown to purple color is produced).

Filter paper is impregnated with the following solutions and dried at 25 degree C.:

Solution 1

2,3-dinitrobenzoic acid (DNBA) 100.0 mg

Potassium hydroxide (KOH) 10.5 g

Borate 30 g

add to 900 mL distilled water, mix, and Q.S. to 1 liter with D.I. water

In this example, a dipstick was prepared in accordance with the instant invention. The DCD device is comprised of a paper carrier matrix impregnated with the composition of solution. Note, the concentrations of any of the above constituents can be varied to suit the device format (e.g. dependent upon paper type, and inclusion of semi-permeable membranes or other innovations utilized in dry chemistry technology).

Production of this test device is carried out using the following procedure. The test device, a piece of Whatman 3 MM filter paper having dimensions of 0.25 inch by 3 inches is impregnated with solution 1 by immersion into it. The paper is then dried by using forced air not exceeding 60 degrees C. The paper is then cut into smaller pieces measuring 0.25 inches by 0.25 inches. The paper is then laminated to one side of a double-sided adhesive transfer tape commercially available from 3M Company, St. Paul, Minn. 55144. This laminate (paper plus adhesive) measures 0.25 inches by 0.25 inches. The laminate is then attached, via the unused adhesive side, to one end of a sturdy polystyrene strip measuring about 0.25 inches by 3 inches; the resulting product forms a test device comprising a 3 inch long polystyrene handle with a square of the impregnated test paper at one end. The dipstick thus obtained will produce a brown to purple color when exposed to creatinine at a concentration of 5 mg/dL creatinine or greater. In fact, the intensity of the color is proportional to the concentration of creatinine present in the sample. The test device, therefore, effectively measures the creatinine concentration of urine providing an accurate method for the normalization of anti-HIV values as well as other clinical markers found in urine.

To summarize Example 1 more specifically, the foregoing dry chemistry test strip (DCD) method to measure the creatinine concentration in a urine sample, the method comprising the steps of preparing a test means by successively impregnating a carrier matrix with reagent solutions, drying said test means, dipping completed test means into a test sample, and determining the quantity of creatinine present in said test sample by comparing the relative intensity and color produced by the reaction to a color chart with color blocks referenced to specific concentrations of creatinine.

The concentrations of constituents in example 6 may be changed and still remain within the scope of the invention and give similar results. The indicator of example 6 can be substituted with any one or more of the following; an antibody to creatinine, an enzyme sensitive to creatinine, any pH sensitive compound, or compound which produces a color or absorbance change in the visible or UV range after undergoing oxidation or reduction. The reactive indicator, DNBA, may be replaced with one or more of the following substitutes with similar chemical reactivity or any creatinine reactive indicator that would fall into the spirit and scope this invention: a (non-explosive) picric acid, anti-creatinine antibody bound to a indicator compound, anti-creatinine antibody bound to a microparticle or other suitable substrate, analogs of DNBA (e.g. 3,4 Dinitro benzoic acid), 2-Naphthol, Naphthol AS, Naphthol AS acetate, Naphthol AS biphosphate, alpha-Naphtholbenzene, 1,2-naphthoquinone, and 1,4-Naphtholquinone. Additionally, the creatinine-reactive indicator, DNBA, may be replaced with an enzyme specific for creatinine including creatinine oxidase, dehydrogenase, amidinohydrolase, or deiminase. For example, the R1 may contain creatinine oxidase, an oxygen acceptor which could be selected from the following group, 4-aminoantipyrine (4AAP), tetramethylbenzidine (TMB), 2,2′-Azino-di-(3-ethylbenzthiazolinesulfonic acid) (ABTS) diammonium salt, or other suitable compound that produces an observable color for the peroxidase/peroxide reaction. Other such compounds may include, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), N,N-Bis(4-sulfobutyl)-3,5-dimethylaniline (MADB), and pyrogallol. Also, 4-aminoantipyrine can be paired with a number of compounds to create a violet to violet-blue color complex in the presence of the peroxide/peroxidase reaction. These compounds include 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. Another indicator pair that may be utilized consists of 3-Methyl-2-benzothiazolinonehydrazone and Dimethylaniline.

In addition, it is possible to conjugate these or other enzymes to antibodies. Consequently, these conjugated pairs can also be substituted into the test reaction together with an appropriate indicator compound. Therefore, this assay may include any antibody or enzyme capable of being conjugated to an antibody.

The buffer and basic compounds in example 6 may be substituted with one or more from the following: citrate, phosphate, phthalate, acetate, hydrochloric acid, nitric acid, phosphoric acid, oxalate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, and succinate. Note: the brand names, trade names, common names, and abbreviations above are commonly used and can be found in the 1999 SIGMA Chemical catalog page 1910. Please note, as revealed previously in this disclosure, surfactants and thickeners are often included in dry chemistry dipstick devices in order to improve accuracy, precision, and color development.

### EXAMPLE 6

The following procedure is a method for manufacturing an aqueous, liquid reagent chemistry test for the determination of anti-HIV in urine on an automated chemistry analyzer or by classical, wet, manual analysis (e.g., with a spectrophotometer).

Reagent Solution 1 (R1):

0.05 M Phosphate buffer pH 7.2

100 nM HIV antigen coated particles

Standard 10 fmol/L anti-HIV Calibrator Solution:

0.05 M Phosphate buffer pH 7.2

10 fmol/L anti-HIV

The reagent system of the instant invention (liquid reagent) is intended for use on any automatic chemistry analyzer with open channel capability including Olympus AU 5000 series, Hitachi 700 series, Beckman CX series and others as commonly known in the art. The reagent is used in the following manner. A method for measuring the anti-HIV (HIV antibody) concentration in order to determine exposure to the AIDS virus (HIV virus) on a test specimen, said test method comprising the steps of placing the reagent composition(s), R-1, in the reagent compartment of the chemistry autoanalyzer, aliquoting samples, calibrators, and controls into sample cups and placing them on the chemistry autoanalyzer, transferring an aliquot (e.g. 10 uL) of each sample, calibrator, and control into single discrete cuvettes mounted within the chemistry autoanalyzer, aliquoting a specified volume (e.g. 250 uL) of the reagent composition of R-1 into each cuvette and mixing, incubating the reaction mixture for a specified time interval, measuring and recording absorbance values of the reaction mixtures with the chemistry autoanalyzer's spectrophotometer at the specified wavelength (e.g. 340 nm) at preprogrammed time intervals, and comparing absorbance values of samples and controls to those of calibrators in the form of a standard curve thereby quantitating the anti-HIV if present. If the sample's absorbance is equal to, or greater than the 10 fmol/L anti-HIV calibrator's absorbance, this indicates a positive value for the presence of anti-HIV which suggest that the individual that gave the urine for testing has been exposed to the AIDS virus; if it is less than the 10 fmol/L anti-HIV then the sample's absorbance will be less than the cutoff calibrator's absorbance will indicate a negative for anti-HIV and no exposure to the HIV virus has occurred. This description of the present art illustrates a completely enabled device that would allow a physician, patient, and/or lab technician to determine the anti-HIV presence in the patient's urine. The normal values for this example are less than 10 fmol/L of anti-HIV indicates normal urine no exposure to the HIV virus, and 10 fmol/L anti-HIV or greater indicates HIV viral exposure.

Changes to the foregoing solutions could be made and still have similar results. The concentrations of said constituents may be changed and still remain within the scope of the invention. Obviously, the same substitution groups for buffers, HIV antigens or antibodies, IgG or use of other antibodies and type and amount of microparticles as noted in Example 1 through 5 also apply to this example.

In the instant invention, when urine is mixed with the reagent system in the prescribed ratio, the anti-HIV concentration will directly affect the absorbance produced by the reaction mixture. Specifically, as the anti-HIV reacts with the conjugated HIV antigen microparticles, agglutination occurs and this antigen-antibody particle agglutination “colony” will absorb and/or reflect light. The comparative absorbance measurements can be made visually or via a spectrophotometer. Note, the vast majority of clinical chemistry analyzers incorporate a spectrophotometer.

Listed below is an example of parameters for the Hitachi 717 analyzer. The settings are intended as guidelines, and are set forth with the understanding that variations may be made to affect performance and still remain within the scope of the invention. Those skilled in the art will recognize that parameters may vary by instrument. Specifications for the Hitachi 717 are as follows:

| Test: | [HIV] |
|---|---|
| say code: | [1 point][40]-[0] |
| Sample volume: | [10][10] |
| R1 volume | [250][100][NO] |
| R2 volume | [ 0][100][NO] |
| Wavelength | [0][340] |
| Calib. Method: | [Linear][0][0] |
| Std. (1) Conc.-POS: | [ 0]-[10]* assigned calibrator value |
| Std. (2) Conc.-POS: | [] - [] |
| Std. (3) Conc.-POS: | [] - [] |
| Std. (4) Conc.-POS: | [] - [] |
| Std. (5) Conc.-POS: | [] - [] |
| Std. (6) Conc.-POS: | [] - [] |
| SD Limit: | [999] |
| Duplicate Limit: | [32000] |
| Sensitivity Limit: | [0] |
| ABS. Limit (INC/DEC): | [32000][INCREASE] |
| Prozone Limit: | [250][upper] |
| Expected Value: | [0]-[10] |
| Tech. Limit: | [0]-[1000] |
| Instrument Factor | [1] |

Please note that dilution of the urine is not required before analysis. This method has a sensitivity of +/−1 fmol/L anti-HIV. The use of a calibrator is not necessary, if a K factor is employed. The K factor can be used in calibrating a method for analysis that utilizes enzymatic or antigen-antibody reactions whose rate of change in absorbance at different concentrations forms a linear plot, and the slope of the plot is already known. The slope is based on the molar absorptivity of the absorbing species (e.g. Naphthol, or NAD) of the chemistry's reaction. The K factor can be calculated as follows; all automated instruments have a K factor mode.

K=total reaction volume (mL)×1000/molar absorptivity×lightpath (cm)×specimen volume (mL)

In K factor calibration, a zero or blank calibrator is run and the absorbance and concentration of this standard, and the predetermined K factor, are used in the calculation of the results of unknown samples.

The automated analysis procedure encompasses the following method for the measurement of anti-HIV on an unknown sample of urine (or other biological sample including serum, whole blood, cerebral spinal fluid, gastric fluid, hair homogenates, sweat extracts, and saliva). To summarize more specifically this example, the foregoing automated method employing an aqueous liquid reagent for measuring the concentration of anti-HIV presence and quantitation in order to determine if anti-HIV is present in a test specimen, said test method comprising the steps of placing the reagent composition(s), R-1, in the reagent compartment of the chemistry autoanalyzer, aliquoting samples, calibrators, and controls into sample cups and placing them on the chemistry autoanalyzer, transferring an aliquot of each sample, calibrator, and control into single, discrete cuvettes mounted within the chemistry autoanalyzer, aliquoting a specified volume of the first reagent composition, R-1, into each cuvette and mixing, incubating the reaction mixture for a specified time interval, measuring and recording absorbance values of the reaction mixtures with the chemistry autoanalyzer's spectrophotometer at the specified monochromatic wavelength (from 340 to 800 nm) at preprogrammed time intervals, and comparing absorbance values of samples and controls to those of the calibrators in the form of a standard curve thereby quantitating the amount anti-HIV if present.

To further improve the accuracy of the present device and eliminate the diurnal effects, the user should perform a creatinine or cystatin C or other renal clearance marker on the same sample of urine. The creatinine assay can be performed on the same autoanalyzer used for the anti-HIV assay and can take the form of the Modified Jaffe method (well known in the art) or other commonly available spectrophotometric assays. Many autoanalyzers will even perform the calculation for the H/C ratio and print it on the test report. Other techniques to produce a creatinine result may be substituted including the DCD and LFD taught herein. Please note, however, that creatinine auto analysis methods typically have a dynamic assay range of 0 to 400 mg/dL. This in combination with the dynamic range of the anti-HIV analysis (i.e. 1 to 1000) will yield a H/C ratio on virtually any random urine sample. Therefore, obtaining an accurate concentration result via instrumental analysis of both creatinine and anti-HIV is an advantage over the semi-quantitative assay ratio obtained using the LFD methods as described herein. This accurate ratio over an extended dynamic range means that no resampling and retesting is required if the creatinine value exceeds 150 mg/dL in the case of a negative anti-HIV result (less than 10 fmol/L of anti-HIV present) because a concentrated urine was tested and validates the negative anti-HIV result, or conversely if the creatinine value is less than 150 mg/dL in the case of a negative anti-HIV result (no anti-HIV is present). This quantitative ratio also provides additional data on the course of the disease. Obviously the higher the ratio the more anti-HIV detected. It is therefore possible to determine if treatment is helping, or not. It is also possible to evaluate the current state of the disease. Please see Examples 1 and 2 for additional information on normalization of random urine anti-HIV and the H/C.

### EXAMPLE 7

The following procedure is a method for manufacturing a dry chemistry test strip (DCD), for the determination of anti-HIV in a test sample.

Filter paper is impregnated with the following solutions and dried at 25 degree C.:

Solution 1

30.2 G PIPES (1,4-Piperazinediethanesulfonic acid)

0.05 Units/mL beta-Galactosidase/HIV antigen (enzyme conjugated to the HIV antigen)

add to 900 mL D.I. water, mix, adjust pH to 6.8, Q.S. to 1000 mL

Solution 2

0.01 M 5-bromo-6-chloro-3-indoxyl-beta-D-galactopyranoside (Magenta- beta-D-Gal)

1 mL (0.1%) DMSO

dissolve in 900.0 mL distilled water, mix, and Q.S. to 1000 mL.

In this example, a dipstick is prepared in accordance with the instant invention as described in Example 5, however, an additional solution is required. This solution 2 is incorporated into the test device by immersing the test paper into solution 2; the paper is then dried by using forced air not exceeding 60 degrees C. If a two-part test pad “sandwich” is used, the pad with solution #1 must be on top and the pad with solution 2 is on the bottom. The dipstick thus obtained will produce a magenta color when exposed to anti-HIV at a concentration of 10 fmol/L or greater. In fact, the intensity of the magenta color is proportional to the concentration of the anti-HIV, present in the sample. This test device, therefore, effectively identifies the presence of anti-HIV in urine by the measurement of the anti-HIV in the urine sample used for illustrative purposes in this example.

To summarize Example 7 more specifically, the foregoing dry chemistry test strip (DCD) method to measure the anti-HIV concentration in a urine sample for the determination of presence of absence of anti-HIV using said sample, the method comprising the steps of preparing a test means by successively impregnating an absorbent carrier matrix with reagent solutions, drying said test means, dipping completed test means into test sample, and determining the quantity of anti-HIV present in said test sample by comparing the relative intensity of the color (magenta) produced by the reaction to a color chart with color blocks referenced to specific concentrations of anti-HIV.

Changes to the foregoing solutions could be made and still have similar results. The foregoing solutions could be combined together, or reduced to include only 1. The concentrations of said constituents may also be changed and still remain within the scope of the invention. The buffer may be replaced with any one or more of those constituents enumerated in Example 1.

The indicator substrate complex in the solution 5-bromo-6-chloro-3-indoxyl-beta-D-galacatopyranoside, could be substituted with one or more of the following: 4-Aminophenyl-beta-D-galactopyranoside, 3-indoxyl-beta-D-galactopyranoside (blue), 5-Bromo-4-chloro-3-indoxyl-beta-D-galactopyranoside (blue), 5-Bromo-3-indoxyl-beta-D-galactopyranoside (blue), 6-chloro-3-indoxyl-beta-D-galactopyranoside (salmon), 6-Fluoro-3-indoxyl-beta-D-galactopyranoside, 8-Hydroxyquinoline-beta-D-galactopyranoside, 5-Iodo-3-indoxyl-beta-D-galactopyranoside (purple), N-Methylindoxyl-beta-D-galactopyranoside, 2-Nitrophenyl-beta-D-galactopyranoside, 4-Nitrophenyl-beta-D-galactopyranoside, Naphthol AS-BI-beta-D-galactopyranoside, and 2-Naphthyl-beta-D-galactopyranoside (yellow). Fluorescent substrates may also be utilized including 4-Methylumbelliferyl-beta-D-glucuronic acid. The colors noted in the parentheses are those produced in the reaction described above. The indicator substrate used in these examples must be matched to the conformation of the galactosidase used (i.e. alpha or beta, and dextrorotorary (D) or levorotorary (L)). For example, beta-D-Galactosidase should be matched with the indicator/substrate Iodo-3-indoxyl-beta-D-galactopyranoside; conversely, alpha-L-Galactosidase would be matched with Iodo-3-indoxyl-alpha-L-galactopyranoside. Note that some cross-reactivity does occur between stereo-isomers and, therefore, it is possible to substitute these compounds where appropriate.

Substitution of the beta-Galactosidase with another enzyme would necessitate a change of substrate indicator complex. If another glycosidase was selected, it would have to be matched to the appropriate substrate (e.g. beta-Cellobiosidase and a cellobioside). Examples of substrates for beta-D-Cellobiosidase include 5-Bromo-4-chloro-3-indoxyl-beta-D-cellobioside, 5-Bromo-6-chloro-3-indoxyl-beta-D-cellobioside, 4-Nitrophenyl-beta-D-cellobioside, 1-Naphthyl-cellobioside, and the fluorescent indicator, 4-Methylumbelliferyl-beta-D-cellobioside.

Other glycosidases which may be substituted for Galactosidase and Cellobiosidase include the alpha and beta, and D and L conformations of the following enzymes: Arabinosidase, Fucosidase, Galactosaminidase, Glucosaminidase, Glucosidase, Glucuronidase, Lactosidase, Maltosidase, Mannosidase, and Xylosidase. Their corresponding substrates, Arabinopyranoside, Fucopyranoside, Galactosaminide, Glucosaminide, Glucopyranoside, Glucuronic acid, Lactopyranoside, Maltopyranoside, Mannopyranoside, and Xylopyranoside may be bound to each of the following color indicator groups: 5-Bromo-4-chloro-3-indoxyl, 5-Bromo-6-chloro-3-indoxyl, 6-chloro-3-indoxyl, 5-Bromo-3-indoxyl, 5-Iodo-3-indoxyl, 3-indoxyl, 2-(6-Bromonaphthyl), 6-Fluoro-3-indoxyl 2-Nitrophenyl, 4-Nitrophenyl, 1-Naphthyl, Naphthyl AS-BI, 2-Nitrophenyl-N-acetyl, 4-Nitrophenyl-N-acetyl, and 4-Methylumbelliferyl moieties.

The glycosidase enzyme conjugated to the HIV antigen in the example above can also be replaced by other types of enzymes whose substrates are compatible with the indicator groups listed above. These include esterases (e.g. Carboxyl esterase, and Cholesterol esterase), sulfatases (e.g. Aryl sufatase), and phosphatases (e.g. Alkaline phosphatase). These enzymes can utilize the indicator groups delineated above when conjugated to the corresponding substrate. For example, Carboxyl esterase and 6-chloro-3-indoxyl butyrate, and Aryl sulfatase and 5-bromo-4-chloro-3-indoxyl sulfate, and Alkaline phosphatase and 2-naphthyl phosphate form enzyme-substrate pairs.

Other enzymes may be conjugated to the HIV antigen, and therefore substituted for the species described above. This group now listed, however, must utilize a substrate that is distinct and separate from the indicator. This enzyme group may include any dehydrogenase, oxidase, hydroxylase, or oxidoreductase. Each grouping will utilize a specific indicator or group of indicators. The dehydrogenases and hydroxylases will utilize a co-enzyme, a color indicator and an electron carrier such as a-NAD (a-Nicotinamide adenine dinucleotide), however this electron carrier/acceptor can be replaced by the alpha or beta isomers of any one of the following substitutes: nicotinamide adenine dinucleotide, nicotinamide adenine dinucleotide 3′-phosphate, nicotinamide adenine dinucleotide phosphate, triphosphopyridine, nicotinamide 1-N1-ethenoadenine dinucleotide phosphate, nicotinamide hypoxanthine dinucleotide, nicotinamide hypoxanthine dinucleotide phosphate, nicotinamide mononucleotide, nicotinamide N1-propylsulfonate, nicotinamide ribose monophosphate, or other analogs of NAD.

Some dehydrogenases and hydroxylases and their substrate pairs which can be used include Formaldehyde dehydrogenase and Formaldehyde, Fructose dehydrogenase and Fructose, Glucose-6-phosphate dehydrogenase and Glucose-6-phosphate, Glucose dehydrogenase and Glucose, Glutamate dehydrogenase and Glutamate, Glycerol dehydrogenase and Glycerol, Glycerol-3-phosphate dehydrogenase and Glycerol-3-phosphate, Hydroxybutyrate dehydrogenase and Hydroxybutyrate, Hydroxybenzoate hydroxylase and 4-Hydroxybenzoate, Lactate dehydrogenase and Lactate, Leucine dehydrogenase and Leucine, Malate dehydrogenase and Malate, Mannitol dehydrogenase and Mannitol, or any other dehydrogenase or hydroxylase.

The use of oxidases to replace the glycosidase also requires a separate indicator, and peroxidase. Some oxidases and their substrate pair which can be used include Acyl-CoA oxidase and Acyl-CoA, Alcohol oxidase and Ethanol, Ascorbate oxidase and Ascorbate, Cholesterol oxidase and Cholesterol, Choline oxidase and Choline, Glucose oxidase and Glucose, Glycerophosphate oxidase and Glycerophosphate, Xanthine oxidase and Xanthine, Uricase and Uric acid, or any other oxidase.

A few color indicators that can be utilized with peroxidase include pyrogallol, ABTS (2,2′-Azinobis(3-ethylbenzthiazoline) sulfonic acid), 3,3′, 5,5′-Tetramethylbenzidine, ortho-Dianisidine, 3,3′-Diaminibenzidine, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), and N,N-Bis(4-sulfobutyl)-3,5-dimethylaniline (MADB). An indicator pair may also be used. One such pair is 3-Methyl-2-benzothiazolinonehydrazone and Dimerhylaniline. Another pair combines 4-aminoantipyrine with a number of compounds to create a violet to violet-blue color complex in the presence of the peroxide/peroxidase reaction. These compounds include phenol, 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. An example of this assay procedure would substitute glucose oxidase for galactosidase in the antibody-enzyme conjugate in R-1; the R-2 would then contain glucose as the substrate and ABTS (reduced) as the indicator. The R-2 would also contain peroxidase, because the product of the reaction between glucose oxidase and glucose yields peroxide. The peroxidase oxidizes any peroxide thus produced, thereby releasing an oxygen atom; this oxygen, in turn, reacts with ABTS, and converts it from the colorless, reduced form to its blue, oxidized form. The intensity of the blue color produced is proportional to the anti-HIV concentration present in the specimen. Clearly, peroxidase may be conjugated to the antibody, and the indicators noted above used with it and its substrate, peroxide.

The use of oxireductases to replace glycosidase also requires a separate indicator including NADPH oxidoreductase and NADPH, or any oxidoreductase. The NADPH oxireductase reduces the NADPH in the presence of Flavin mononucleotide (FMN). This reaction may be observed visually by utilizing the same color indicators as delineated for the dehydrogenases, or measured spectrophotometrically at 340 nm.

The antigens used in this example and the prior examples may be substituted with any one or more of the following anti-HIV (I or II), HIV antigens (I or II), anti-IgG, anti-IgM or other human antibodies or HIV aptamers. All of these reactants can be used and will produce a detectable response in the presence of HIV antibody.

### EXAMPLE 8

The following procedure is a method for manufacturing a dry chemistry test strip, (DCD) for the determination of anti-HIV in a test sample by measurement of its Anti-HIV concentration. Filter paper is impregnated with the following solutions and dried at 25 degree C.:

Solution 1

2-[N-Morpholino]ethansulfonic Acid buffer (MES) 0.1 M

HIV antigen is conjugated to horseradish peroxidase

900 mL D.I. water, mix, adjust pH to 6.0, and Q.S. to 1000 mL with D.I. water

Solution 2

2-[N-Morpholino]ethansulfonic Acid buffer 0.1 M

Tetramethylbenzidine, (TMB) 500 mg

Urea-Peroxide, 5.0 g

900 mL D.I. water, mix, and adjust pH between 5.0 and 7.0, preferably 6.0

Q.S. to 1000 mL with D.I. water

Antibodies conjugated to horseradish peroxidase can be obtained from Biodesign International; the techniques for producing these types of conjugated antibodies is also well known in the art.

This assay utilizes an antigen/antibody reaction with the antibody conjugated to peroxidase. When antibody which is conjugated to the peroxidase binds to its target antigen, it releases the peroxidase which is then free to react with peroxide and the chromogen, TMB, resulting in formation of a blue-green colored complex. This color reaction yields a visible color change. Therefore, the anti-HIV concentration is proportional to the intensity of the blue-green color produced.

The test device in this example is manufactured in the same manner as that in Example 9. If this device is constructed using two reaction pads, the reaction pad containing solution 2 must be on the bottom half of the “sandwich”. In addition, it may be necessary to separate the two pads with a semipermeable membrane.

Changes to the foregoing solutions could be made and still have similar results. The foregoing solutions could be combined together, or reduced to only 1. The concentrations of said constituents may also be changed and still remain within the scope of the invention. Obviously, the same substitution groups for anti-HIV and HIV antigens are possible as already demonstrated in examples 1-7 and this includes the buffers as noted in the prior examples also apply to this example. The urea peroxide was chosen, because it is more stable than simple peroxide. It is obvious, however, that one may utilize any peroxide-containing compound to act as a substrate to peroxidase.

The TMB may be replaced by any suitable compound that will produce an observable color as part of the peroxidase/peroxide reaction. Other such compounds include ABTS (2,2′-Azino-di-(3-ethylbenzthiazolinesulfonic acid) diammonium salt, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl- N-(2-hydroxy-3-sulfopropyl)aniline (ALOS), N-Ethyl-N-(3 -sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), N,N-Bis(4-sulfobutyl)-3,5-dimethylaniline (MADB), and pyrogallol. Also, 4-aminoantipyrine can be paired with a number of compounds to create a violet to violet-blue color complex in the presence of the peroxide/peroxidase reaction. These compounds include 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. Another indicator pair that may be utilized consists of 3-Methyl-2-benzothiazolinonehydrazone and Dimerhylaniline.

In addition, it is possible to conjugate other enzymes to antibodies or antigens. Consequently, these conjugated pairs can also be substituted into the test reaction together with an appropriate indicator compound. Therefore, this assay may include any enzyme capable of being conjugated to an antibody or antigen.

To further describe the preferred test method for determining the presence of anti-HIV by the measurement of anti-HIV in an unknown test sample, the assay system can take the form of a dipstick (DCD), lateral flow device (LFD), or an aqueous liquid reagent that is composed of a buffer and an indicator that produces a color or change in the intensity of color or absorbance in the UV or visible spectrum in the presence of anti-HIV. The antibodies (such as anti-HIV, anti-anti-HIV, anti-IgG or others), antigens (i.e. HIV antigens or recombinant HIV antigens), and HIV aptamers are usable as taught. The anti-IgG human antibodies can also include IgA, IgD, IgE, and IgM. The buffers used may be any one or more compounds selected from the following group and enumerated by their common names: citrate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, phosphate, phthalate, succinate, hydrochloric acid, sulfuric acid, nitric acid, acetic acid, sodium hydroxide, and potassium hydroxide. In addition, as taught the test sample can be any biological fluid from the following group: urine, serum, whole blood, saliva, cerebral spinal fluid, gastric contents, and extracts of hair or sweat. This art as taught herein can employ an aqueous-based liquid reagent for measuring the concentration of anti-HIV in order to determine if the individual that is giving the urine specimen for testing has been exposed to the AIDS (HIV) virus, said test method comprising the steps of placing the reagent in the reagent compartment of the chemistry autoanalyzer, aliquoting samples, calibrators, and controls into sample cups and placing them on the chemistry autoanalyzer, transferring an aliquot of each sample, calibrator, and control into single, discrete cuvettes mounted within the chemistry autoanalyzer, aliquoting a specified volume of the reagent composition into each cuvette and mixing, incubating the reaction mixture for a specified time interval, measuring and recording absorbance values of the reaction mixtures with the chemistry autoanalyzer's spectrophotometer at the specified wavelength (from 340 to 800 nm) at preprogrammed time intervals, and comparing absorbance values of samples and controls to those of the calibrators in the form of a standard curve thereby quantitating the anti-HIV if present. The above described assay method also is applied to creatinine, cystatin C or other renal clearance markers for determine of urine sample concentration.

This art as taught in previous examples can also employ a dry chemistry test strip (DCD) method for measuring the anti-HIV concentration in a test sample, the method comprising the steps of preparing a test means by successively impregnating a carrier matrix with reagent solutions, drying said test means, dipping completed test means into test sample, and determining the quantity of anti-HIV present in said test sample by comparing the relative intensity of the color produced by the reaction to a color chart with color blocks referenced to specific concentrations of anti-HIV. The above described assay method may also be applied to creatinine or cystatin C or other renal clearance marker determination.

This art as taught in previous examples can also employ a dry chemistry, lateral flow device (LFD) for measuring the anti-HIV concentration in a test sample, the method comprising the steps of preparing a test means by successively impregnating a solid, absorbent carrier matrix with liquid, reagent solutions at specific locations on the test means, drying said test means, dipping completed test means into test sample or pipetting test sample onto the test means, and determining the quantity of anti-HIV in the test sample by comparing the relative intensity (completeness) of the assay line produced by the reaction to a standard chart, or by comparing the relative intensity (completeness) of the assay line produced by the reaction to the control line. The above described assay method may also be applied to creatinine, cystatin C, or other renal clearance marker determination.

These methods as taught for the measurement of anti-HIV can be used in conjunction with assay methods for determining the creatinine, cystatin C or other renal clearance marker concentration of a test sample and using the determined concentration to normalize the sample via the anti-HIV/creatinine, cystatin C or other renal clearance marker ratio (H/C ratio) for more accurate evaluation of anti-HIV in the patient's test sample. The specific gravity or cystatin C may also be used for this purpose. This disclosure, therefore, describes a method for determining the creatinine, cystatin C, or other renal clearance marker concentration of a test sample and using said creatinine or other marker concentration to normalize the sample for accurate determination of anti-HIV present.

The subject invention provides an extraordinary and novel method for quantitating the presence of anti-HIV in a biological specimen (i.e. urine, blood, serum, saliva, hair and sweat extracts, and cerebrospinal fluid) in order to determine if the individual presenting/giving the sample for testing has been exposed to the HIV virus.

In addition, the absolute novelty of creatinine, cystatin C, or other renal clearance marker measurement by the use of a DCD or LFD is of enormous value to medical diagnostics and the health of our population.; its utility when applied to aqueous, liquid form and modified for use on automated clinical chemistry analyzers is also of great value for the same reasons. All in all, the ability of the present art to analyze urine for antiO-HIV measurement via DCD, LFD, and aqueous, liquid reagent while simultaneously enabling the user to normalize the results with the sample's creatinine, cystatin C or other renal clearance marker concentration as described herein is a substantial and significant improvement over the prior art.

To further elaborate the present art so that it is clearly understood the present art is a method for determining the presence of HIV antibodies (anti-HIV) on an unknown test sample, said test method being composed of a buffer, antibody or antigen or indicator that produces a detectable response or a change in the absorbance or intensity of a color or line in the UV or visible spectrum in the presence or absence of anti-HIV. This is a method wherein the antibody or antigen to anti-HIV can be selected from the group consisting of anti-HIV (I or II), anti-anti-HIV, HIV antigens (I or II), recombinant HIV antigens, HIV aptamers, anti-Human IgG, IgA, IgD, IgE, or IgM. The methods buffer can be can be any one or more compounds selected from the group consisting of and enumerated by their common names; citrate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, phosphate, phthalate, succinate, hydrochloric acid, sulfuric acid, nitric acid, acetic acid, sodium hydroxide, and potassium hydroxide. It is understood that the method's as taught can use test samples from any biological fluid from the following group: urine, serum, whole blood, saliva, cerebral spinal fluid, gastric contents, and extracts of hair or sweat. These methods can use all the buffers, indicators, microparticles (metallic or other matrix), and components as taught in examples 1 through 8. The methods as taught employ aqueous liquid reagents for measuring the concentration of anti-HIV on a test specimen, said test methods comprise the steps of placing the reagent in the reagent compartment of the chemistry autoanalyzer and aliquoting samples, calibrators, and controls into sample cups and placing them on the chemistry autoanalyzer, then transferring an aliquot of each sample, calibrator, and control into single, discrete cuvettes mounted within the chemistry autoanalyzer, aliquoting a specified volume of the reagent composition into each cuvette and mixing, incubating the reaction mixture for a specified time interval, and measuring and recording absorbance values of the reaction mixtures with the chemistry autoanalyzer's spectrophotometer at the specified wavelength (from 340 to 800 nm) at preprogrammed time intervals, and comparing absorbance values of samples and controls to those of the calibrators in the form of a standard curve thereby quantitating the amount of anti-HIV present. The methods as taught can also employ a dry chemistry test strip (DCD) method to measure the anti-HIV concentration in a test sample, the method comprising the steps of preparing a test means by successively impregnating an absorbent carrier matrix with reagent solutions, drying said test means, dipping completed test means into test sample, and determining the quantity of anti-HIV present in said test sample by comparing the relative intensity of the color produced by the reaction to a color chart with color blocks referenced to specific concentrations of anti-HIV. The methods can also employ a dry chemistry lateral flow device (LFD) for measuring the anti-HIV concentration in a test sample, the method comprising the steps of preparing a test means by successively impregnating a solid, absorbent carrier matrix with liquid reagent solutions at specific locations on said test means, drying said test means, dipping completed test means into test sample or pipetting test sample onto the test means, and determining the quantity of anti-HIV present in said test sample by comparing the relative intensity of the assay line produced by the reaction to a standard chart, or by comparing the relative intensity of the assay line produced by the reaction to the control line. The method examples as taught utilizing a spectrophotometer can employ wavelengths from 340 to 800 nm. The methods as the present art teaches can also improve analytical value of the anti-HIV concentration of a test sample by employing creatinine, cystatin C, or specific gravity concentrations which can be used to normalize the sample for accurate determination of anti-HIV. This normalization of the anti-HIV concentration requires that it be divided by the creatinine, cystatin C, or specific gravity concentration of the same test sample thereby yielding the anti-HIV to creatinine, cystatin C, or specific gravity ratio. Thus, all the methods of the present art as taught are for analyzing a sample using a dry chemistry dipstick or lateral flow device, or aqueous liquid reagent to determine the concentration of HIV antibody in an individual's random urine sample in order to determine if the individual's exposure to the HIV virus, and normalizing or correcting this assay value with the sample's creatinine, cystatin C, or specific gravity concentration.

I claim:

1. A method according to claim 1 employing an aqueous liquid reagent for measuring the concentration of anti-HIV on a test specimen, said test method comprising the steps of;
(a) placing the reagent in the reagent compartment of the chemistry auto analyzer,
(b) aliquoting samples, calibrators, and controls into sample cups and placing them on the chemistry autoanalyzer,
(c) transferring an aliquot of each sample, calibrator, and control into single, discrete cuvettes mounted within the chemistry autoanalyzer,
(d) aliquoting a specified volume of the reagent composition into each cuvette and mixing,
(e) incubating the reaction mixture for a specified time interval,
(f) measuring and recording absorbance values of the reaction mixtures with the chemistry autoanalyzer's spectrophotometer at the specified wavelength (from 340 to 800 nm) at preprogrammed time intervals,
(g) and comparing absorbance values of samples and controls to those of the calibrators in the form of a standard curve thereby quantitating the amount of anti-HIV present.

2. The method according to claim 1 wherein the spectrophotometric wavelength employed is from 340 to 800 nm.

3. The method according to claim 1 for determining the anti-HIV concentration of a test sample wherein creatinine, cystatin C, or specific gravity concentration of the specimen can be used to normalize the specimen for accurate determination of anti-HIV present in the specimen.

4. The method according to claim 1 wherein the reagent composition is composed of buffer and HIV antigen coated particles.

5. The method according to claim 4 wherein the buffer can be selected from the group consisting of citrate, hepes, tris (trizma), taps, popso, tes, pipes, mopso, tricine, mops, mes, bicine, bes, caps, epps, dipso, ches, capso, ampso, aces, ada, bis-tris-propane, tapso, heppso, tea, amp, phosphate, phthalate, succinate, hydrochloric acid, sulfuric acid, nitric acid, acetic acid, sodium hydroxide, and potassium hydroxide.

6. The method according to claim 4 wherein the HIV antigen can be substituted from the group consisting of HIV antigens (I or II), anti-IgG, anti-IgM or other human antibodies.
